(19) United States
(12) Patent Application Publication
(10) Pub. No.: US 2011/0288389 A9
(43) Pub. Date: Nov. 24, 2011

(54) OXYGEN SENSOR

(75) Inventors: Douglas A. Levinson, Sherborn, MA (US); Howard Bernstein, Cambridge, MA (US)

(73) Assignee: Seventh Sense Biosystems, Inc., Cambridge, MA (US)

(21) Appl. No.: 12/716,222

(22) Filed: Mar. 2, 2010

Related U.S. Application Data
(60) Provisional application No. 61/156,632, filed on Mar. 2, 2009.
(60) Provisional application No. 61/163,710, filed on Mar. 26, 2009.
(60) Provisional application No. 61/269,436, filed on Jun. 24, 2009.
(60) Provisional application No. 61/257,731, filed on Nov. 3, 2009.

(51) Int. Cl. A61B 5/145

(52) U.S. Cl. 600/364

(57) ABSTRACT

The present invention generally relates to systems and methods for determining oxygen in a sample, or in a subject. In one aspect, the present invention is generally directed to an article exhibiting a determinable feature responsive to oxygen, such as oxygen-sensitive particles. The particles may exhibit a determinable change with a change in oxygen concentration, and such particles can accordingly be used to determine oxygen. For example, in one set of embodiments, the particles may be at least partially coated with a protein, such as hemoglobin, that is able to interact with oxygen. In some cases, the protein may aggregate under certain conditions (e.g., under relatively low oxygen concentrations), and such protein aggregation may be used, for example, to cause the particles to become aggregated, which can be determined in some way. In some cases, such aggregation may be irreversible; i.e., the degree of aggregation corresponds to the most extreme oxygen concentrations that the proteins were exposed to. Such articles may be used, for example, to determine oxygen within a sample, or within a subject, such as a human subject. For instance, the article may be formed as a skin patch, or administered to the skin of a subject, e.g., on the surface of the skin, within the dermis or epidermis, etc., to determine oxygen within the subject.

### RELATED APPLICATIONS

This application claims the benefit of U.S. Provisional Patent Application Ser. No. 61/163,710, filed Mar. 26, 2009, entitled “Systems and Methods for Creating and Using Suction Blisters or Other Pooled Regions of Fluid within the Skin,” by Levinson, et al.; U.S. Provisional Patent Application Ser. No. 61/156,632, filed Mar. 2, 2009, entitled “Oxygen Sensor,” by Levinson, et al.; U.S. Provisional Patent Application Ser. No. 61/269,436, filed Jun. 24, 2009, entitled “Devices and Techniques associated with Diagnostics, Therapies, and Other Applications, Including Skin-Associated Applications,” by Levinson, et al.; and U.S. Provisional Patent Application Ser. No. 61/257,731, filed Nov. 3, 2009, entitled “Devices and Techniques associated with Diagnostics, Therapies, and Other Applications, Including Skin-Associated Applications,” by Bernstein, et al. Each of the above is incorporated herein by reference.

### FIELD OF INVENTION

The present invention generally relates to systems and methods for determining oxygen in a sample, or in a subject.

### BACKGROUND

The blood delivers vital oxygen from the lungs to the cells of the body. However, various medical conditions are characterized by low levels of oxygen within the blood (hypoxemia) or within the body (hypoxia). Under such conditions, tissues within the body are deprived of adequate oxygen supply, and such tissues may be temporarily or permanently harmed as a result. Such conditions may arise, for example, due to sleep apnea, pressure ulcers or blisters, bed sores, or in certain infants. Systems and methods for the determination of oxygen within a subject are thus of great importance.

### SUMMARY OF THE INVENTION

The present invention generally relates to systems and methods for determining oxygen in a sample, or in a subject. The subject matter of the present invention involves, in some cases, interrelated products, alternative solutions to a particular problem, and/or a plurality of different uses of one or more systems and/or articles.

In one aspect, the present invention is directed to an article. According to one set of embodiments, the article includes a skin delivery device able to selectively determine localized oxygen within one of the dermis, the epidermis, the interstitial fluid, or the blood within the skin when the skin delivery device is applied to a subject.

In another set of embodiments, the article includes a device at least partially insertable into the skin of a subject. In some cases, the device is able to determine oxygen concentration of or proximate at least a portion of the skin of the subject.

The article, in yet another set of embodiments, includes a skin patch exhibiting a determinable feature responsive to oxygen when the skin patch is applied to a subject. In still another set of embodiments, the article includes a skin delivery device containing a plurality of agents that exhibit increasing aggregation with decreasing oxygen concentration.

In accordance with yet another set of embodiments, the article includes a plurality of particles at least partially coated with sickle-cell hemoglobin. The article, in another set of embodiments, includes a skin delivery device containing a plurality of particles at least partially coated with a hemoglobin.

In one set of embodiments, the article includes a liquid containing a plurality of agents that are able to aggregate when the concentration of oxygen within the liquid is less than about 90% of the saturation oxygen concentration of the liquid, but are not able to substantially aggregate when the liquid is saturated with oxygen.

The article, in accordance with another set of embodiments, includes a liquid containing a plurality of particles coated with a polymer that exhibits at least about 10% polymerization when the concentration of oxygen within the liquid is less than about 90% of the saturation oxygen concentration of the liquid, but is not able to substantially polymerize when the liquid is saturated with oxygen.

In still another set of embodiments, the article includes a plurality of particles coated with a polymer that exhibits at least about 10% polymerization when exposed to blood containing a concentration of oxygen less than about 90% of the saturation oxygen concentration of the blood.

In another aspect, the present invention is directed to a device able to determine localized oxygen proximate the skin when the device is applied to the skin of a subject.

The invention, in yet another aspect, is directed to a method. In one set of embodiments, the method includes an act of determining blood oxygen in a subject by administering an oxygen-sensitive agent to the subject.

According to another set of embodiments, the method includes an act of determining blood oxygen in a subject by applying a skin patch to the subject.

The method, in yet another set of embodiments, includes an act of administering a plurality of particles at least partially coated with hemoglobin to the skin of a subject. According to still another set of embodiments, the method includes an act of determining a region on the skin of a subject having low oxygen, relative to the oxygen of the blood of the subject, by applying a skin patch to the subject. In yet another set of embodiments, the method includes an act of determining a region on the skin of a subject having low oxygen, relative to the blood of the subject, by administering a plurality of particles to the skin of the subject.

In one set of embodiments, the method includes an act of diagnosing a subject suspected or at risk of having sleep apnea by determining a determinable feature of a skin patch applied to the subject prior to the subject sleeping. In another set of embodiments, the method includes an act of diagnosing a subject suspected or at risk of having sleep apnea by determining a determinable feature of a plurality of particles applied to the skin of the subject.

The method, in still another set of embodiments, includes an act of applying an oxygen-sensitive agent to a tissue.

In another aspect, the present invention is directed to a method of making one or more of the embodiments described herein, for example, an article exhibiting a determinable feature responsive to oxygen. In another aspect, the present invention is directed to a method of using one or more of the embodiments described herein, for example, an article exhibiting a determinable feature responsive to oxygen.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates one embodiment of the invention comprising a patch applied to the skin of a subject;

FIG. 2 illustrates another embodiment of the invention in which particles are administered into the skin of a subject;

FIGS. 3A-3B illustrate the aggregation of particles having first and second regions, according to yet another embodiment of the invention;

FIGS. 4A-4B illustrate devices according to certain embodiments of the invention;

FIGS. 5A-5C illustrate devices according to various embodiments of the invention;

FIG. 5D illustrates a kit containing more than one device, in yet another embodiment of the invention; and

FIG. 5E illustrates a device according to still another embodiment of the invention.

### DETAILED DESCRIPTION

The present invention generally relates to systems and methods for determining oxygen in a sample, or in a subject. The invention provides, in some embodiments, compositions and devices to be positioned on, in, or proximate the skin of a subject, which can determine oxygen levels associated with skin, blood, and/or interstitial fluid, and/or which delivers a signal indicative of oxygen level. As described more fully below, the signal can be visual, or another sensory signal such as a stimulus affecting feel, smell, taste, or the like, or a signal readable by an instrument. The signal can be readable by the subject in which oxygen level determination is being made, or by another person or machine, or other entity. In several embodiments, devices of the invention function by including one or more agents which can react with or otherwise be affected by oxygen, or by another species in a subject that can be used to determine oxygen level. In most of the description herein, the agents are particles that are functionalized so as to interact with the species in the subject, for example by clustering and providing a different visual appearance relative to a non-clustered state in the absence of the species. Other particle behaviors and signaling techniques are provided below. It is to be understood that wherever particles are described as useful in devices and to techniques of the invention, other agents, as described generally below, can be substituted.

In one aspect, devices of the invention are provided that can monitor oxygen level within a subject either continuously, or at any discrete point in time, or both. For example, a device can be constructed to provide constant, real-time signal production indicative of oxygen level, or as another example, a device that changes color, or color intensity, reversibly and/or essentially instantaneously in reaction to skin oxygen contents. Or, a device of the invention can be constructed to measure oxygen at a particular point in time and “hold” the signal indicative of that oxygen content at that point in time, for example, a point in time during the night when a subject is sleeping. Or, a device can be constructed to measure and report a highest-level and/or lowest-level oxygen content; a device can be applied to the skin and at the end of a determination period (for example, overnight) report the lowest oxygen level of the subject during that measuring period of time. As a non-limiting example, in some embodiments, the device may include agents such as particles that interact with oxygen, and can exhibit aggregation as a function oxygen concentration, reversibly and/or irreversibly; this aggregation may be determined to determine oxygen levels within a subject.

Species that can interact with agents (e.g., particles) and devices of the invention to measure and report oxygen content can be natural bodily-occurring species, non-naturally occurring species that are added to a subject, or the like. In some cases, devices and compositions of the invention can be provided in the form of skin-adhesive patches, implants, devices otherwise held proximate the skin (on or in, for example, lotion, clothes, and/or other personally proximate objects such as bandages, jewelry, stocking, etc.).

The above introductory description outlines, generally, various aspects of the invention. More details of various aspects and embodiments are provided below.

In one aspect, the present invention is generally directed to an article exhibiting a determinable feature responsive to oxygen, such as oxygen-sensitive particles. The particles may exhibit a determinable change with a change in oxygen concentration, and such particles can accordingly be used to determine oxygen. For example, in one set of embodiments, the particles may be at least partially coated with a protein, such as hemoglobin, that is able to interact with oxygen. In some cases, the protein may aggregate under certain conditions (e.g., under relatively low oxygen concentrations), and such protein aggregation may be used, for example, to cause the particles to become aggregated, which can be determined in some way. In some cases, such aggregation may be irreversible; i.e., the degree of aggregation corresponds to the most extreme oxygen concentrations that the proteins were exposed to. Such articles may be used, for example, to determine oxygen within a sample, or within a subject, such as a human subject. For instance, the article may be formed as a skin patch, or administered to the skin of a subject, e.g., on the surface of the skin, within the dermis or epidermis, etc., to determine oxygen within the subject.

Thus, various aspects of the present invention are generally directed to systems and methods for determining oxygen amounts or concentrations in a sample, or in a subject. Such determinations of oxygen may be quantitative, and/or qualitative in some cases, e.g., the determination may be that the amount or concentration of oxygen within a sample has increased or decreased in some fashion, or that there are sufficient or insufficient levels of oxygen present. “Determine,” in this context, generally refers to the analysis of a species such as oxygen, for example, quantitatively or qualitatively, and/or the detection of the presence or absence of the species. The species may be, for example, a bodily fluid and/or an analyte suspected of being present in the bodily fluid. For instance, the concentration or the amount of oxygen may be determined. “Determining” may also refer to the analysis of an interaction between two or more species, for example, quantitatively or qualitatively, and/or by detecting the presence or absence of the interaction, e.g. determination of the binding between oxygen and another species, such as is discussed below.

Certain embodiments are directed to the determination of amounts or concentrations of oxygen in a subject, such as a human subject. In some cases, however, the subject may be a non-human animal. Examples of such subjects include, but are not limited to, a mammal such as a dog, a cat, a horse, a rabbit, a cow, a pig, a sheep, a goat, a rat (e.g., Rattus Norvegicus), a mouse (e.g., Mus musculus), a guinea pig, a hamster, a primate (e.g., a monkey, a chimpanzee, a baboon, an ape, a gorilla, etc.), a bird, a reptile, a fish, or the like. In certain cases, as discussed below, the oxygen concentration within the skin of the subject is determined in some fashion. For example, a skin delivery device or a skin patch may be applied to the skin of a subject, and used to determine oxygen within the subject, e.g., within the skin or within the blood of the subject, depending on placement.

However, in other embodiments, the amount or concentration of oxygen within a sample may be determined, for example, a biological sample or a chemical sample such as a solution or a liquid. As non-limiting examples, in one embodiment, the amount or concentration of oxygen within a tissue sample or an isolated organ may be determined In another example, the amount or concentration of oxygen within a reactor may be determined In yet another example, the amount or concentration of oxygen within a food, a drug or a pharmaceutical preparation, or a consumer item may be determined.

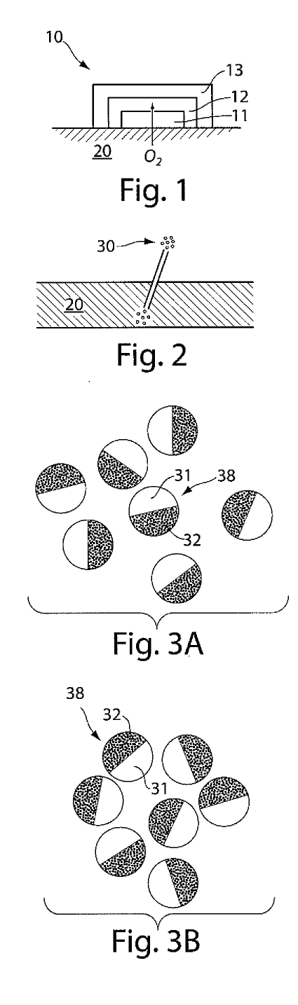

As a non-limiting example, an embodiment of the invention, as used to determine the oxygen concentration within the skin of a subject, is now described with reference to the schematic diagram shown in FIG. 1 (not to scale). In this figure, skin patch 10 is disposed on the surface of the skin 20 of a subject. Typically, a skin patch includes one or more layers of material that are adhered to the surface of the skin. For instance, in this example, skin patch 10 includes a first adhesive layer 11, which is used to affix the patch to the surface of the skin; a detection layer 12 for determining oxygen concentrations or amounts; and a selectively impermeable layer 13 to prevent the detection layer from being exposed to the external environment (e.g., containing atmospheric oxygen, water, etc.) surrounding the patch. In this example, oxygen from skin 20 is able to pass through adhesive layer 11 into detection layer 12; thus, adhesive layer 11 may be formed from an oxygen-permeable material, or adhesive layer 11 may be sufficiently porous to allow oxygen transport to occur therethrough, etc. In contrast, selectively impermeable layer 13 may be sufficiently impermeable to oxygen such that detection layer 12 is not significantly affected by the concentration of oxygen outside of selectively impermeable layer 13 (although in some cases, selectively impermeable layer 13 may be permeable to species other than oxygen, i.e., the impermeability of layer 13 is determined with reference to oxygen, not to other species). Thus, “selectively impermeable” layer, as used herein, means that the layer that is essentially impermeable to a species that would affect the technique adversely (e.g., oxygen), but which may or may not be permeable to other species, e.g., to other species that do not adversely affect the assay or other technique of the invention. “Essentially” impermeable, in this context, means that the layer resists permeability to species that would adversely affect the assay or other technique, e.g., within the time frame of the assay or other technique.

Detection layer 12, in this example, includes an agent that exhibits a determinable change when exposed to different concentrations or amounts of oxygen, i.e., the agent is one that is “oxygen-sensitive.” For instance, in one set of embodiments, detection layer 12 may contain a plurality of particles that are at least partially coated with a protein, such as hemoglobin, that is able to interact with oxygen. For example, the protein may be one that can aggregate or polymerize under certain conditions, such as under relatively low oxygen concentrations. In one embodiment, the hemoglobin may be a sickle cell hemoglobin or other modified hemoglobin that shows increased sensitivity to oxygen, relative to unmodified hemoglobin. By controlling the amount of protein coated on the particle, e.g., by controlling the location and/or concentration of protein on the surface of the particle, the amount of sensitivity of the particles to oxygen may be controlled. Thus, for example, the particles may exhibit substantially no aggregation when exposed to normal concentrations of oxygen (for example, normal concentrations of atmospheric oxygen, normal concentrations of dissolved oxygen within the blood), but the particles may exhibit some aggregation as the concentration of oxygen decreases, e.g., below a certain threshold concentration. As discussed below, the detection of such particles, e.g., within detection layer 12, may be achieved due to aggregation of the particles (e.g., causing a difference in appearance, color, light scattering, etc.), or in some cases, the particles, when aggregated may produce a determinable signal, e.g., a change in temperature, or color. It should be noted that detection layer 12, in this example, is able to detect oxygen in or proximate the skin, as oxygen diffusing across the skin into the device may be determined as outlined above.

As another non-limiting example, FIG. 2 illustrates another embodiment of the invention where oxygen-sensitive particles 30, such as those discussed above, are administered or delivered directly to the skin 20 of a subject. In some cases, the particles may be administered to any suitable location within the skin of the subject, e.g., to the epidermis, dermis, or below the dermis in some cases. In one embodiment the particles are administered to a suction blister, as discussed below. Such particles may, for example, exhibit relatively little or no aggregation when exposed to normal concentrations of oxygen (e.g., within the skin or blood of the subject), but exhibit some aggregation at lower concentrations of oxygen. By determining the degree of aggregation, the concentration of oxygen may be determined.

As discussed, several aspects of the present invention are directed to varying agents that exhibits a determinable change when exposed to different concentrations or amounts of oxygen. Such agents may, in some cases, be contained within suitable articles, which can be delivered to a sample, or to a subject. Non-limiting examples of an oxygen-sensitive agent include particles, such as anisotropic particles, that exhibit oxygen sensitivity. Other examples of oxygen-sensitive agents include, but are not limited to, polymers that exhibit different degrees of polymerization when exposed to different oxygen concentrations, dyes or other entities sensitive to oxygen, methelyne blue, or 2,6-dichlorophindophenol. Methylene blue or 2,6-dichlorophindophenol are generally colorless until oxidized in the presence of O₂. For instance, under exposure to at least 30 mmHg O₂, methylene blue or 2,6-dichlorophindophenol may change colors, indicating the presence of oxygen. The response time for such dyes may be between 20-30 s to 1 hour in some cases.

Accordingly, certain embodiments of the invention are directed to oxygen-sensitive particles that may be delivered to a sample, or to a subject. The particles may include microparticles and/or nanoparticles in some cases. The particles may be chosen, in some embodiments, to be relatively non-toxic or non-reactive, i.e., to the sample or to the subject, depending on the application, and examples of compositions of such particles are discussed in detail below.

In one set of embodiments, the particles may be rendered oxygen-sensitive by using an agent that exhibits a determinable change when exposed to different concentrations or amounts of oxygen, for example, a change in aggregation, polymerization, or the like. The agent may be formed as part of the particle or homogenously contained within the particle, or in some cases, the agent may be one that is coated on at least a portion of the surface of the particle, for instance, covalently attached to the surface of the particle.

For example, at least a portion of the particle may be functionalized, i.e. comprising surface functional moieties, to which a protein or other agent may be bound to, thereby coating at least a portion of the surface of the particle with the protein or other agent. The functional moieties may include simple groups, selected from the groups including, but not limited to, —OH, —CHO, —COOH, —SO₃H, —CN, —NH₂, —SH, —COSH, —COOR, or halide; biomolecular entities including, but not limited to, amino acids, proteins, sugars, DNA, antibodies, antigens, and enzymes; grafted polymer chains, selected from a group of polymers including, but not limited to, polyamide, polyester, polyimide, polyacrylic; a thin coating covering the surface of the particle, including, but not limited to, the following groups of materials: metals, semiconductors, and insulators, which may be a metallic element, an oxide, an sulfide, a nitride, a selenide, a polymer, a polymer gel, or the like. The protein or other agent can then be reacted with the functional moiety, e.g., using a suitable cross-linking reagent, such as glutaradehyde.

In one set of embodiments, the agent is a protein that is able to interact with oxygen, and in some cases, the protein may bind to oxygen, e.g., through coordination chemistry. Non-limiting examples of such proteins include hemoglobin, myoglobin, hemocyanin, hemerythrin, chlorocruorin, vanabin, erythrocruorin, pinnaglobin, leghemoglobin, etc. The protein may be human or from another species. If the protein is hemoglobin, the hemoglobin may be adult hemoglobin (e.g. human hemoglobin A) or fetal hemoglobin (e.g., human hemoglobin F). Upon interaction with oxygen, the protein may exhibit a conformational change, and/or a change in a physical property, that alters the ability of the protein to interact with other species, e.g., with other proteins or ligands. Thus, as an example, oxyhemoglobin (hemoglobin bound to oxygen) may exhibit a first affinity to a surface (e.g., to another particle), while deoxyhemoglobin (hemoglobin free of oxygen) may exhibit a second affinity to the surface, and thus, the surface may have different concentrations of particles adsorbed thereon, depending on the concentration of oxygen.

In one set of embodiments, the protein may be sickle cell hemoglobin, where the hemoglobin contains one or more mutations that causes the hemoglobin to agglomerate or polymerize in solution, in some cases forming fibers or other aggregates. For instance, one form of sickle cell hemoglobin has a sequence that is the same as that of normal (wild-type) hemoglobin except that glutamic acid in position 6 (in the beta chain) has been mutated to valine. This mutation allows the deoxygenated form of the sickle cell hemoglobin to polymerize, and the degree of polymerization is dependent, at least in part, on the concentration of oxygen. Other mutations to hemoglobin, not necessarily in patients having sickle cell anemia, may also have exhibit similar polymerization or aggregation tendencies. Accordingly, one embodiment of the invention comprises a plurality of particles containing a sickle-cell hemoglobin, for example, such that the surfaces are at least partially coated with sickle-cell hemoglobin. Sickle-cell hemoglobins can be obtained commercially from a number of different suppliers, obtained from patients exhibiting symptoms of sickle-cell anemia, or synthesized using techniques known to those of ordinary skill in the art.

Other agents that exhibit a determinable change when exposed to different concentrations or amounts of oxygen may be used in certain embodiments of the invention. For example, in one set of embodiments, a monomer such as ethene that is able to polymerize to form polymers may be used. In certain cases, oxygen concentrations may at least partially inhibit certain types of polymerization reactions such as free-radical chain polymerization, as oxygen may inhibit such reactions by consuming free radicals, thereby limiting the free-radical polymerization reaction. Thus, the degree of polymerization exhibited by such agents may be related to the concentration of oxygen present, and particles coated with such monomers may be allowed to polymerize to determine the concentration of oxygen present. As another example of a suitable agents, certain compounds may be used that are sensitive to dissolved oxygen concentrations, such as tris(4,4′-diphenyl-2,2′-bipyridine) ruthenium (II) chloride pentahydrate, methylene blue, or 2,6-dichlorophindophenol. In some embodiments, such agents may be contained within particles, e.g., on the surface and/or within the particles. Particles containing such agents, e.g., within and/or on their surfaces, may be delivered to a sample, or to a subject, and a determinable feature (e.g., color) may be determined to determine oxygen amounts or concentrations, e.g., in, on, or proximate to the skin of the subject.

Particles containing such agents may, in certain instances, exhibit polymerization or aggregation that is a function of the amount or concentration of oxygen surrounding the particles, e.g., contained within a gas or liquid (water, blood, interstitial fluid, media, etc.) surrounding the particles. For instance, the amount of polymerization or aggregation may increase (or decrease) with decreasing oxygen concentration, depending on the embodiment, and the amount of polymerization or aggregation can be controlled in some cases by controlling the concentration or location of protein within and/or on the surface of the particles. Such particles can be readily optimized for a particular application, using routine optimization techniques and the like. For instance, the polymerization or aggregation of such particles may be controlled such that the particles are not able to aggregate when the surrounding liquid (e.g., aqueous solution, blood, interstitial fluid, etc., depending on the application) is saturated with oxygen, but are able to aggregate when the concentration of oxygen is less than about 95%, less than about 90%, about 80% or about 70% of this value, e.g., at least about 10% or about 20% of the particles are able to aggregate or polymerize under such conditions.

In some cases, the aggregation or polymerization is irreversible, for example, as with certain sickle cell hemoglobins or polymerization reactions. Such irreversibly-aggregating particles may be useful in certain applications, for example, to determine the most extreme oxygen concentrations that the particles were exposed to. Thus, for instance, particles may be applied to a sample, or to a subject, and then analyzed at a later point in time (e.g., the following day) to determine the lowest oxygen concentrations the particles were exposed to during that time interval. As specific examples, such information may be useful for subjects having or at risk for pressure blisters, bed sores, or the like, or in applications where the particles are shipped with another material where information regarding oxygen exposure is desirable (e.g., food, organs for transplant, or the like).

Particles that have aggregated or polymerized may be determined using any suitable technique, for example, via a change in an optical property (e.g., color), a change in the temperature of the particles, a change in an electrical property of the particles, etc. In some cases, the change may be one that is determinable by a human, unaided by any equipment that may be directly applied to the human. For instance, the determinable change may be a change in appearance (e.g., color), a change in temperature, a change in sensation, the production of an odor, etc. In other cases, however, the aggregation or polymerization may be determined using suitable equipment or assays.

In some embodiments, multiple particles, when aggregated, may become visible, e.g., as discrete aggregates and/or as a change in color. In another example, the aggregates themselves may not be visible, but an optical property of the medium containing the aggregates may be altered in some fashion (e.g., exhibiting different light scattering properties, different opacities, different degrees of transparency, etc.), which can be determined to determine oxygen. In some cases, as discussed below, the aggregation of particles may bring two or more reaction entities contained on or in the particles into close proximity, and the reactants may react in some fashion that can be determined, e.g., by producing light, producing heat, etc. In cases where a reaction entity is present, e.g., on the surface (or at least a portion of the surface) of the particle, the reaction entity may be any entity able to interact with and/or associate with an analyte (e.g., oxygen), or another reaction entity, for example, chemically and/or physically. For instance, the reaction entity may be a binding partner able to bind an analyte. For example, the reaction entity may be a molecule that can undergo binding with a particular analyte, for example proteins such as those described herein.

In some cases, the aggregates may precipitate and/or flocculate. For instance, if the particles are present in a solution, the aggregates may separate from the solution, and optionally can be removed or otherwise analyzed. As additional examples, other properties of the particles may be determined to determine oxygen, e.g., a change in a chemical property of the particles, a change in the appearance and/or optical properties of the particles, a change in the temperature of the particles, a change in an electrical property of the particles, etc. In some cases, the change may be one that is determinable by a human, unaided by any equipment that may be directly applied to the human. For instance, the determinable change may be a change in appearance (e.g., color), a change in temperature, the production of an odor, etc., which can be determined by the human eye without the use of any equipment.

One example of an embodiment that uses a change in color is now discussed. In some cases, the particles may comprise a first surface region and a second surface region. The first surface region may, for instance, have a first color and the second region may have a second color, where the first surface region is coated with an oxygen-sensitive agent that exhibits increasing aggregation with decreasing oxygen concentration. At normal oxygen concentrations, the particles remain largely unaggregated, thereby giving the appearance of a blend of the first color and the second color, as is illustrated in FIG. 3 with particles 38 having a first region 31 and a second region 32, which are randomly distributed in this figure. However, at lower concentrations of oxygen, some of the particles may aggregate, and aggregate such that the first regions of the particles become oriented towards each other, e.g. due to the presence of the oxygen-sensitive agent on first region 31 but not on second region 32. As this occurs, the second color of the particles may dominate over the first color as the particles aggregate. Accordingly, in this example, the concentration of oxygen may be determined by determining color.

Other properties may also be determined besides color. Accordingly, it should be understood that the use of “color” with respect to particles as used herein is by way of example only, and other properties may be determined instead of or in addition to color. For instance, aggregation of particles may cause a change in an electrical or a magnetic property of the particles, which can be determined by determining an electrical or a magnetic field. For example, an aggregate of particles may have a different magnetic moment than isolated particles, which can be determined by determining a magnetic property of the particles. As another example, the particles may contain a first region and a second region having different reactivities (e.g., the first region may be reactive to an enzyme, an antibody, etc.), and aggregation of the particles may cause a net change in the reactivity. As still another example, size may be used. For instance, the aggregates may be visually identifiable, the aggregates may form a precipitant, or the like. Thus, for example, the particles (which may be anisotropic or not anisotropic) may appear to be a first color when separate, and a second color when aggregation occurs. In some cases, an assay (e.g., an agglutination assay) may be used to determine the aggregation.

A non-limiting example of how an aggregate of particles may produce a chemical reaction follows. In one embodiment, there may be first particles containing a first reaction entity and a second reaction entity that reacts with the first reaction entity; when the particles aggregate, the first and second reaction entities may react. As a specific example, the reaction between the first and second reaction entities may be an endothermic or an exothermic reaction; thus, when the particles are brought together, a temperature change is produced, which can be determined in some fashion. Thus, the reaction between the first and second reactants can be induced or at least accelerated by brining the particles closer together. The first and second reactants may be any suitable reactants. For instance, the first and second reactants can produce heat (e.g., as in an exothermic reaction), cold (e.g., as in an endothermic reaction), a change in color, a product which can then be determined, or the like. As another example, a reaction between the first and second reactants may cause the release of a material. In some cases, the material may be one that can be sensed by a subject, e.g., capsaicin, an acid, an allergen, or the like. Thus, the subject may sense the change as a change in temperature, pain, itchiness, swelling, taste, or the like. Examples of suitable capsaicin and capsaicin-like molecules include, but are not limited to, dihydrocapsaicin, nordihydrocapsaicin, homodihydrocapsaicin, homocapsaicin, or nonivamide.

A non-limiting example of a change in temperature follows. The first particle may contain barium hydroxide (Ba(OH)₂), while the second particle may contain ammonium nitrate (NH₄NO₃). The particles may be present in solution or suspension, and only a low level of reaction between the barium hydroxide and the ammonium nitrate occurs. Each particle may also contain an oxygen-sensitive agent that exhibits increasing aggregation with decreasing oxygen concentration. When oxygen is added, aggregation of the particles may occur due to this agent. As the particles aggregate, the reaction rate between the reactants may increase as the particles are brought closer to each other. In this case, the reaction between barium hydroxide and the ammonium nitrate is an endothermic reaction that yields barium nitrate (Ba(NO₃)₂) and ammonium (NH₃). This reaction may be determined by determining a drop in temperature.

In some cases, the first and second reactants may each be present on the same particles, but the reactants on the same particles may not be able to react with each other, e.g., due to spatial separation on the particles. However, when aggregated, different particles may come into contact with each other, thereby allowing a reaction to occur.

In one aspect, particles or other agents that exhibits a determinable change when exposed to different concentrations or amounts of oxygen may be administered to a subject, e.g., to determine amounts or concentrations of oxygen within the subject. In some cases, the determination may localized, e.g., to the skin, to a specific region within the skin, and/or to a specific region of the body. For example, depending on where the particles or other agents are delivered, the oxygen concentration determined may be that of the dermis, the epidermis, the interstitial fluid, the blood, etc. In some cases, e.g., during surgical procedures or the like, the particles (or other agents) may be desirably administered to other locations, tissues, or organs within a subject. For example, the particles may be administered to the heart during cardiac surgery to monitor the condition of the heart during the operation.

It should be understood, however, that the application of the particles or other agents to a subject, such as a human subject, is by way of example only, and in other cases, the particles or other agents may be applied to other samples, which may be biological or non-biological. For instance, the oxygen concentration within a tissue sample, an isolated organ, a liquid sample, or the like may be determined using the systems and methods described herein, e.g., by applying the particles or other agents to such samples.

In certain embodiments, a skin delivery device can be used to administer the particles or other agents to a subject. For instance, the skin delivery device may be a handheld device that is applied to the surface of the skin of a subject. In some cases, the device may be sufficiently small or portable that the subject can self-administer the device. In certain instances, the device may be applied to the surface of the skin, and is not inserted into the skin. In other embodiments, however, at least a portion of the device may be inserted into the skin, for example, mechanically. For example, in one embodiment, the device may include a cutter, such as a hypodermic needle, a knife blade, a piercing element (e.g., a solid needle), or the like. In some cases, the device may be designed such that portions of the device are separable. For example, a first portion of the device may be removed from the surface of the skin, leaving other portions of the device behind on the skin Such devices may be useful, for instance, for determining oxygen amounts or concentrations, on, or proximate to the skin of the subject.

In one set of embodiments, the skin delivery device may take the form of a skin patch. Typically, a skin patch includes one or more layers of material that are adhered to the surface of the skin, and can be applied by the subject or another person. In certain embodiments, layers or portions of the skin patch may be removed, leaving other layers or portions behind on the skin. Often, the skin patch lacks an external power source, although the various layers of the patch may contain various chemicals, such as drugs, therapeutic agents, diagnostic agents, reaction entities, etc. In some cases, the skin patch may also include mechanical elements as well, for example, a cutter, such as a hypodermic needle, a knife blade, a piercing element (e.g., a solid needle), or the like.

As discussed, in some cases, the skin patch may contain an adhesive layer, a detection layer, and a selectively impermeable layer, e.g., as is shown in FIG. 1. In some embodiments, other materials and/or layers within the patch may be present in addition to these layers. The adhesive layer may be used to affix the patch to the surface of the skin, and may comprise any suitable adhesive, e.g., temporary or permanent. For example, the adhesive layer may comprise pressure sensitive adhesives including polyisobutylene, polystyrene-block-polyisoprene-block-polystyrene, polysiloxane adhesives, polyacrylic adhesives, or the like. The adhesive layer may be at least partially oxygen permeable, e.g., such that at least some oxygen is able to be transported through the adhesive layer, and in some cases, at a transport rate greater than the characteristic detection rate of the portion of the detection layer that is able to determine the oxygen transported therethrough, i.e., the adhesive layer does not present a significant transport barrier to oxygen, and the ability of the detection layer to determine oxygen is not substantially statistically different when the adhesive layer is present versus when the adhesive layer is absent. In some cases, the adhesive layer itself is inherently at least partially oxygen permeable, e.g., due to the composition of the adhesive layer. In other cases, the adhesive layer may comprise a relatively oxygen-impermeable material, but the adhesive layer may nonetheless be sufficiently oxygen permeable due to its structure. For example, the adhesive layer may have one or more holes that allow oxygen to pass therethrough, or the adhesive layer may be porous, etc.

The detection layer within the skin patch may comprise an oxygen-sensitive agent that exhibits a determinable change when exposed to different concentrations or amounts of oxygen, for instance, oxygen-sensitive particles such as those described herein. Thus, the oxygen-sensitive agent may exhibit a determinable property that is a function of the oxygen concentration or amount, for example, an optical property (e.g., color), temperature, sensation, odor, pain, itchiness, swelling, taste, etc., as previously described.

The patch may also contain, in some cases, a selectively impermeable layer, which can be used to isolate the detection layer from the outside environment surrounding the patch. Thus, the oxygen concentration or amount determined by the detection layer may be a function of the oxygen within the subject (e.g., within the skin of the subject), instead of being contaminated or conflated with environmental oxygen. It should be noted, however, that the selectively impermeable layer may be permeable to species other than oxygen, in some cases. Examples of suitable materials that may be used to form the selectively impermeable layer include, but are not limited to, polypropylene, low-density polyethylene, or other suitable materials. As used herein, a material is sufficiently oxygen impermeable when the transport rate of oxygen through the selectively impermeable layer is at least one order of magnitude less than the transport rate of oxygen through the adhesive layer (if present) or otherwise from the subject to the detection layer. Thus, the oxygen impermeable material need not be one that is perfectly oxygen impermeable.

In another set of embodiments, the particles or other agents may be administered directly to the skin, e.g., to the surface of the skin, to the bloodstream, etc. If the particles are delivered to the skin of the subject, the particles may be delivered to any location within the skin (or below the skin), e.g., to the epidermis, to the dermis, subcutaneously, intramuscularly, etc. In some cases, a “depot” of particles may be formed within the skin, and the depot may be temporary or permanent. For instance, the particles within the depot may eventually degrade (e.g., if the particles are biodegradable), enter the bloodstream, or be sloughed off to the environment. As an example, if the particles are delivered primarily to the epidermis, many of the particles can eventually be sloughed off to the environment (as the epidermis is sloughed off), i.e., such that the particles are present within the subject on a temporary basis (e.g., on a time scale of days or weeks). However, if the particles are delivered to lower layers of tissue, e.g., to the dermis or lower, then the particles may not be as readily sloughed off to the environment (or the particles may take longer to be sloughed off into the environment), and thus the particles may be present in the skin on a longer basis. For instance, the particles may be present within the subject for weeks, months, or years.

In some cases, especially if the particles are colored, the particles after delivery may give the appearance of a “tattoo” or a permanent mark within the skin, and the tattoo or other mark may be of any color and/or size. However, as discussed, other properties besides color may be determined, e.g., temperature changes, chemical reactions (e.g., capsaicin), or the like.

The particles may be delivered to the skin using any suitable technique, and various techniques for delivery into various layers of the skin (or below the skin) are well-known to those of ordinary skill in the art. In many cases, the particles may be dissolved and/or suspended in a carrying fluid or liquid, e.g., saline, or the particles may be contained within a matrix, e.g., a porous matrix that is or becomes accessible by interstitial fluid or blood after delivery. For instance, the matrix may be formed from a biodegradable and/or biocompatible material such as polylactic acid, polyglycolic acid, poly(lactic-co-glycolic acid), etc., or other similar materials.

In some cases, the matrix may prevent or at least inhibit an immunological response by the subject to the presence of the particles, while allowing equilibration of oxygen, etc. with the particles to occur, e.g., if the matrix is porous. For instance, the pores of a porous matrix may be such that immune cells are unable to penetrate. The pores may be, for instance, less than about 5 micrometers, less than about 4 micrometers, less than about 3 micrometers, less than about 2 micrometers, less than about 1.5 micrometers, less than about 1.0 micrometers, less than about 0.75 micrometers, less than about 0.6 micrometers, less than about 0.5 micrometers, less than about 0.4 micrometers, less than about 0.3 micrometers, less than about 0.1 micrometers, less than about 0.07 micrometers, and in other embodiments, or less than about 0.05 micrometers. The matrix may comprise, for example, biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these, and/or other materials such as agarose, collagen, fibrin, or the like.

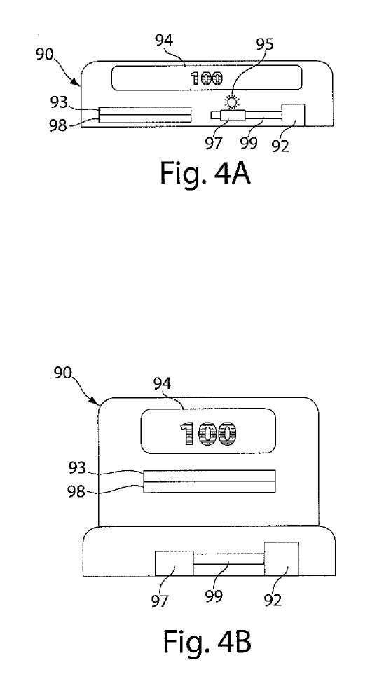

Other non-limiting examples of various devices of the invention are shown in FIG. 4. In FIG. 4A, device 90 is used for withdrawing a fluid from a subject when the device is placed on the skin of a subject. Device 90 includes sensor 95 and fluid transporter 92, e.g., a needle, a microneedle, etc., as discussed herein. In fluidic communication with fluid transporter 92 via fluidic channel 99 is sensing chamber 97. In one embodiment, sensing chamber 97 may contain particles, or another agent that exhibits a determinable change when exposed to different concentrations or amounts of oxygen. In some cases, fluid may be withdrawn using fluid transporter 92 by a vacuum, for example, a self-contained vacuum contained within device 90. Optionally, device 90 also contains a display 94 and associated electronics 93, batteries or other power supplies, etc., which may be used to display sensor readings obtained via sensor 95. In addition, device 90 may also optionally contain memory 98, transmitters for transmitting a signal indicative of sensor 95 to a receiver, etc.

In some cases, a fluid transporter may be used to transport an agent into the skin, and/or transport a fluid out of the skin. As used herein, “fluid transporter” is any component or combination of components that facilitates movement of a fluid from one portion of the device to another. For example, at or near the skin, a fluid transporter can be a hollow needle when a hollow needle is used or, if a solid needle is used, then if fluid migrates along the needle due to surface forces (e.g., capillary action), then the solid needle can be a fluid transporter. If fluid (e.g. blood or interstitial fluid) partially or fully fills an enclosure surrounding a needle after puncture of skin (whether the needle is or is not withdrawn from the skin after puncture), then the enclosure can define a fluid transporter. Other components including partially or fully enclosed channels, microfluidic channels, tubes, wicking members, vacuum containers, etc. can be fluid transporters

In the example shown in FIG. 4A, device 90 may contain a vacuum source (not shown) that is self-contained within device 90, although in other embodiments, the vacuum source may be external to device 90. (In still other instances, other systems may be used to deliver and/or withdraw fluid from the skin, as is discussed herein.) In one embodiment, after being placed on the skin of a subject, the skin may be drawn upward into a recess containing fluid transporter 92, for example, upon exposure to the vacuum source. Access to the vacuum source may be controlled by any suitable method, e.g., by piercing a seal or a septum; by opening a valve or moving a gate, etc. For instance, upon activation of device 90, e.g., by the subject, remotely, automatically, etc., the vacuum source may be put into fluidic communication with the recess such that skin is drawn into the recess containing fluid transporter 92 due to the vacuum. Skin drawn into the recess may come into contact with fluid transporter 92 (e.g., solid or hollow needles), which may, in some cases, pierce the skin and allow a fluid to be delivered and/or withdrawn from the skin. In another embodiment, fluid transporter 92 may be actuated and moved downward to come into contact with the skin, and optionally retracted after use.

Another non-limiting example of a device is shown in FIG. 4B. This figure illustrates a device useful for delivering a fluid to the subject. Device 90 in this figure includes fluid transporter 92, e.g., a needle, a microneedle, etc., as discussed herein. In fluidic communication with fluid transporter 92 via fluidic channel 99 is chamber 97, which may contain particles or other agents that exhibits a determinable change when exposed to different concentrations or amounts of oxygen. These may be delivered to the subject. In some cases, fluid may be delivered with a pressure controller, and/or withdrawn using fluid transporter 92 by a vacuum, for example, a self-contained vacuum contained within device 90. For instance, upon creating a vacuum, skin may be drawn up towards fluid transporter 92, and fluid transporter 92 may pierce the skin Fluid from chamber 97 can then be delivered into the skin through fluid channel 99 and fluid transporter 92. Optionally, device 90 also contains a display 94 and associated electronics 93, batteries or other power supplies, etc., which may be used control delivery of fluid to the skin. In addition, device 90 may also optionally contain memory 98, transmitters for transmitting a signal indicative of device 90 or fluid delivery to a receiver, etc.

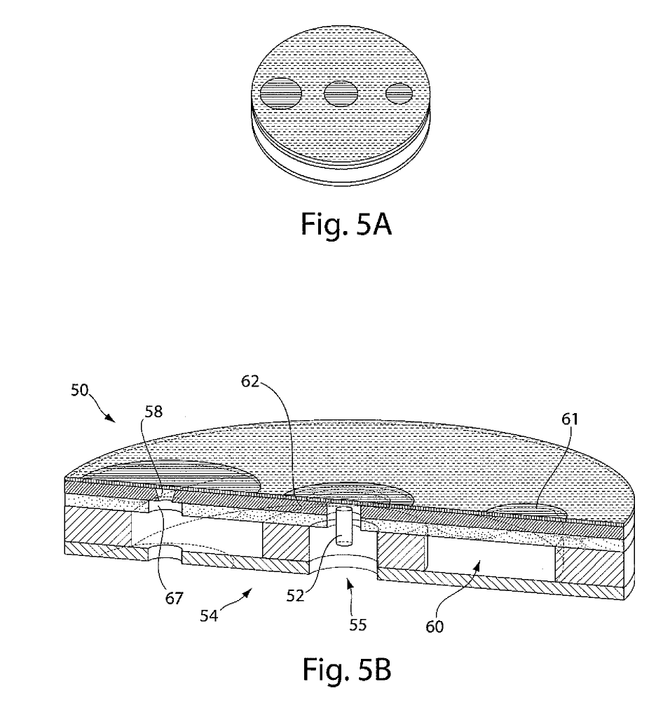

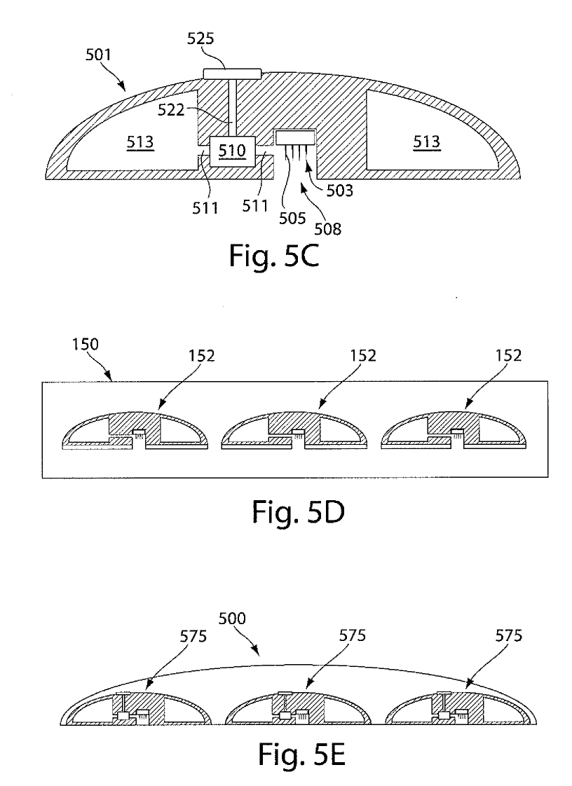

Yet another non-limiting example of a device of the invention is shown in FIG. 5. FIG. 5A illustrates a view of the device (with the cover removed), while FIG. 5B schematically illustrates the device in cross-section. In FIG. 5B, device 50 includes a needle 52 contained within a recess 55. Needle 52 may be solid or hollow, depending on the embodiment. Device 50 also includes a self-contained vacuum chamber 60, which wraps around the central portion of the device where needle 52 and recess 55 are located. A channel 62 connects vacuum chamber 60 with recess 55, separated by a foil or a membrane 67. Also shown in device 50 is button 58. When pushed, button 58 breaks foil 67, thereby connecting vacuum chamber 50 with recess 55, thereby creating a vacuum in recess 55. The vacuum may be used, for example, to draw skin into recess 55, preferably such that it contacts needle 52 and pierces the surface, thereby gaining access to an internal fluid. The fluid may be controlled, for example, by controlling the size of needle 52, and thereby the depth of penetration. For example, the penetration may be limited to the epidermis, e.g., to collect interstitial fluid, or to the dermis, e.g., to collect blood. In some cases, the vacuum may also be used to at least partially secure device 50 on the surface of the skin, and/or to assist in the withdrawal of fluid from the skin. For instance, fluid may flow into channel 62 under action of the vacuum, and optionally to sensor 61, e.g., for detection of oxygen contained within the fluid (such as by exposure to particles or other agents that exhibits a determinable change when exposed to different concentrations or amounts of oxygen). For instance, sensor 61 may produce a color change if oxygen is present (e.g., due to agglomeration), or otherwise produce a detectable signal.

Other components may be added to the example of the device illustrated in FIG. 5, in some embodiments of the invention. For example, device 50 may contain a cover, displays, ports, transmitters, sensors, microfluidic channels, chambers, fluid channels, and/or various electronics, e.g., to control or monitor fluid transport into or out of device 50, to determine oxygen in the subject, to determine the status of the device, to report or transmit information regarding the device and/or analytes, or the like, as is discussed in more detail herein. As another example, device 50 may contain an adhesive, e.g., on surface 54, for adhesion of the device to the skin.

Yet another non-limiting example is illustrated with reference to FIG. 5C. In this example, device 500 includes a support structure 501, and an associated fluid transporter system 503. Fluid transporter system 503 includes a plurality of microneedles 505, although other fluid transporters as discussed herein may also be used. Also shown in this figure is sensor 510, connected via channels 511 to recess 508 containing needles or microneedles 505. Chamber 513 may be a self-contained vacuum chamber, and chamber 513 may be in fluidic communication with recess 508 via channel 511, for example, as controlled by a controller or an actuator (not shown). In this figure, device 500 also contains display 525, which is connected to sensor 510 via electrical connection 522. As an example of use of device 500, when fluid is drawn from the skin (e.g., blood, interstitial fluid, etc.), the fluid may flow through channel 511 to be determined by sensor 510, e.g., due to action of the vacuum from vacuum chamber 513. In some cases, the vacuum is used, for example, to draw skin into recess 508, e.g., such that it contacts needles or microneedles 505 and pierces the surface of the skin to gain access to a fluid internal of the subject, such as blood or interstitial fluid, etc. The fluid may be controlled, for example, by controlling the size of needle 52, and thereby the depth of penetration. For example, the penetration may be limited to the epidermis, e.g., to collect interstitial fluid, or to the dermis, e.g., to collect blood. Upon determination of the fluid and/or an analyte present or suspected to be present within the fluid, a microprocessor or other controller may display on display 525 a suitable signal. It should be noted that a display is shown in this figure by way of example only; in other embodiments, no display may be present, or other signals may be used, for example, lights, smell, sound, feel, taste, or the like.

In some cases, more than one fluid transporter system may be present within the device. For instance, the device may be able to be used repeatedly, and/or the device may be able to deliver and/or withdraw fluid at more than one location on a subject, e.g., sequentially and/or simultaneously. In some cases, the device may be able to simultaneously deliver and withdraw fluid to and from a subject. A non-limiting example of a device having more than one fluid transporter system is illustrated with reference to FIG. 5E. In this example, device 500 contains a plurality of structures such as those described herein for delivering to and/or withdrawing fluid from a subject. For example, device 500 in this example contains 3 such units, although any number of units are possible in other embodiments. In this example, device 500 contains three such fluid transporter systems 575. Each of these fluid transporter systems may independently have the same or different structures, depending on the particular application, and they may have structures such as those described herein.

In another set of embodiments, the device may comprise a mechanism able to cut or pierce the surface of the skin. The cutter may comprise any mechanism able to create a path to a pooled region of fluid through which fluids may be delivered and/or withdrawn from the pooled region. For example, the cutter may comprise a hypodermic needle, a blade (e.g., a knife blade, a serrated blade, etc.), a piercing element (e.g., a solid or a hollow needle), or the like, which can be applied to the skin to create a suitable conduit for the withdrawal of fluid from the pooled region of fluid from the skin In one embodiment, a cutter is used to create such a pathway and removed, then fluid may be delivered and/or withdrawn via this pathway. In another embodiment, the cutter remains in place within the skin, and fluid may be delivered and/or withdrawn through a conduit within the cutter.

In some cases, a hypodermic needle or similar device may be used to deliver particles into various tissues. Hypodermic needles are well-known to those of ordinary skill in the art, and can be obtained with a range of needle gauges. As another example, microneedles such as those disclosed in U.S. Pat. No. 6,334,856, issued Jan. 1, 2002, entitled “Microneedle Devices and Methods of Manufacture and Use Thereof,” by Allen, et al., may be used to deliver the particles to the dermis and/or the epidermis, depending on the shape and/or size of the microneedles, as well as the location of delivery. The microneedles may be formed from any suitable material, e.g., metals, ceramics, semiconductors, organics, polymers, and/or composites. Examples include, but are not limited to, pharmaceutical grade stainless steel, gold, titanium, nickel, iron, gold, tin, chromium, copper, alloys of these or other metals, silicon, silicon dioxide, and polymers, including polymers of hydroxy acids such as lactic acid and glycolic acid polylactide, polyglycolide, polylactide-co-glycolide, and copolymers with polyethylene glycol, polyanhydrides, polyorthoesters, polyurethanes, polybutyric acid, polyvaleric acid, polylactide-co-caprolactone, polycarbonate, polymethacrylic acid, polyethylenevinyl acetate, polytetrafluorethylene, or polyesters. In some cases, the particles may be delivered via the microneedles; in other cases, however, the microneedles may be first applied to the skin and removed to create passages through the skin (e.g., through the stratum corneum, which is the outermost layer of the skin), then the particles subsequently applied to the skin.

As still another example, pressurized fluids may be used to deliver the particles, for instance, using a jet injector or a “hypospray.” Typically, such devices produce a high-pressure “jet” of liquid or powder (e.g., a biocompatible liquid, such as saline) that drives the particles into the skin, and the depth of penetration may be controlled, for instance, by controlling the pressure of the jet. The pressure may come from any suitable source, e.g., a standard gas cylinder or a gas cartridge. A non-limiting example of such a device can be seen in U.S. Pat. No. 4,103,684, issued Aug. 1, 1978, entitled “Hydraulically Powered Hypodermic Injector with Adapters for Reducing and Increasing Fluid Injection Force,” by Ismach.

As yet another example, the particles may be contained within a cream or a lotion which can be rubbed onto the skin to deliver the particles. The cream or lotion may contain, for instance, an emulsion of a hydrophobic and a hydrophilic material (e.g., oil and water), distributed in any order (e.g., oil-in-water or water-in-oil), and the particles may be present in any one or more of the emulsion phases. In some cases, the particles may be administered by a medical practitioner; in other cases, however, the particles may be self-administered.

In one set of embodiments, one or more skin insertion objects may be used to deliver the particles. The skin insertion objects can be constructed to deliver the particles to the dermis and/or to the epidermis, depending on the specific application. The skin insertion objects may be constructed such that at least a portion of the objects is inserted into the skin and include a plurality of particles (or other objects) that, when the skin insertion objects are delivered into the skin, are released into the skin. Accordingly, the skin insertion objects may have any suitable shape that allows this to occur, e.g., having the shape of a solid or a hollow needle, which may be cylindrical or may be tapered, etc. For instance, the particles may be fastened to the skin insertion objects with a degree of adhesion such that, when the skin insertion objects are delivered, at least a portion of the particles remain in the dermis and/or epidermis when the skin insertion objects are removed, e.g., due to friction. As another example, a portion of the skin insertion objects may break off upon entry into the skin, thereby delivering the particles. As mentioned, in some cases, one or more skin insertion objects may be present, e.g., immobilized relative to a substrate for simultaneous delivery. The skin insertion objects may be formed out of any suitable material, including biocompatible and/or biodegradable materials such as those described herein. In other cases, however, the skin insertion objects are formed from other materials that are not necessarily biocompatible and/or biodegradable. The skin insertion objects may be delivered to the skin manually, or in some cases, with the aid of a device.

In yet another set of embodiments, fluid may be delivered and/or withdrawn using an electric charge. For example, reverse iontophoresis may be used. Without wishing to be bound by any theory, reverse iontophoresis uses a small electric current to drive charged and highly polar compounds across the skin. Since the skin is negatively charged at physiologic pH, it may act as a permselective membrane to cations, and the passage of counterions across the skin can induce an electroosmotic solvent flow that may carry neutral molecules in the anode-to-cathode direction. Components in the solvent flow may be analyzed as described elsewhere herein. In some instances, a reverse iontophoresis apparatus may comprise an anode cell and a cathode cell, each in contact with the skin The anode cell may be filled, for example, with an aqueous buffer solution (i.e., aqueous Tris buffer) having a pH greater than 4 and an electrolyte (i.e. sodium chloride). The cathode cell can be filled with aqueous buffer. As one example, a first electrode (e.g., an anode) can be inserted into the anode cell and a second electrode (e.g., a cathode) can be inserted in the cathode cell. In some embodiments, the electrodes are not in direct contact with the skin.

A current may be applied to induce reverse iontophoresis, thereby extracting a fluid from the skin. The current applied may be, for example, greater than 0.01 mA, greater than 0.3 mA, greater than 0.1 mA, greater than 0.3 mA, greater than 0.5 mA, or greater than 1 mA. It should be understood that currents outside these ranges may be used as well. The current may be applied for a set period of time. For example, the current may be applied for greater than 30 seconds, greater than 1 minute, greater than 5 minutes, greater than 30 minutes, greater than 1 hour, greater than 2 hours, or greater than 5 hours. It should be understood that times outside these ranges may be used as well.

In one set of embodiments, the device may comprise an apparatus for ablating the skin. Without wishing to be bound by any theory, it is believed that ablation comprises removing a microscopic patch of stratum corneum (i.e., ablation forms a micropore), thus allowing access to bodily fluids. In some cases, thermal, radiofrequency, and/or laser energy may be used for ablation. In some instances, thermal ablation may be applied using a heating element. Radiofrequency ablation may be carried out using a frequency and energy capable of heating water and/or tissue. A laser may also be used to irradiate a location on the skin to remove a portion. In some embodiments, the heat may be applied in pulses such that a steep temperature gradient exists essentially perpendicular to the surface of the skin. For example, a temperature of at least 100° C., at least 200° C., at least 300° C., or at least 400° C. may be applied for less than 1 second, less than 0.1 seconds, less than 0.01 seconds, less than 0.005 seconds, or less than 0.001 seconds.

As yet another example, the particles may be delivered to a suction blister or other pooled regions of fluid within the skin. In one set of embodiments, a pooled region of fluid can be created between the dermis and epidermis of the skin Such regions can be created by causing the dermis and the epidermis to at least partially separate, and a number of techniques can be used to at least partially separate the dermis from the epidermis.

Pooled regions of fluids, if present, may be formed on any suitable location within the skin of a subject. Factors such as safety or convenience may be used to select a suitable location, as (in humans) the skin is relatively uniform through the body, with the exception of the hands and feet. As non-limiting examples, the pooled region may be formed on an arm or a leg, on the hands (e.g., on the back of the hand), on the feet, on the chest, abdomen, or the back of the subject, or the like. The size of the pooled region of fluid that is formed in the skin and/or the duration that the pooled region lasts within the skin depends on a variety of factors, such as the technique of creating the pooled region, the size of the pooled region, the size of the region of skin to which the technique is applied, the amount of fluid removed from the pooled region (if any), any materials that are delivered into the pooled region, or the like. For example, if vacuum is applied to the skin to create a suction blister, the vacuum applied to the skin, the duration of the vacuum, and/or the area of the skin affected may be controlled to control the size and/or duration of the suction blister. In some embodiments, it may be desirable to keep the pooled regions relatively small, for instance, to prevent an unsightly visual appearance, to allow for greater sampling accuracy (due to a smaller volume of material), or to allow for more controlled placement of particles within the skin For example, the volume of the pooled region may be kept to less than about 2 ml, less than about 1 ml, less than about 500 microliters, less than about 300 microliters, less than about 100 microliters, less than about 50 microliters, less than about 30 microliters, less than about 10 microliters, etc., in certain cases, or the average diameter of the pooled region (i.e., the diameter of a circle having the same area as the pooled region) may be kept to less than about 5 cm, less than about 4 cm, less than about 3 cm, less than about 2 cm, less than about 1 cm, less than about 5 mm, less than about 4 mm, less than about 3 mm, less than about 2 mm, or less than about 1 mm.

A variety of techniques may be used to cause pooled regions of fluid to form within the skin and/or to withdraw a bodily fluid from the skin of a subject such as interstitial fluid or blood. In one set of embodiments, vacuum is applied to create a suction blister. In other embodiments, however, other methods may be used to create as a pooled region of fluid within the skin and/or withdraw fluid from the skin besides, or in addition to, the use of vacuum. When vacuum (i.e., the amount of pressure below atmospheric pressure, such that atmospheric pressure has a vacuum pressure of 0 mmHg, i.e., the pressure is gauge pressure rather than absolute pressure) is used to at least partially separate the dermis from the epidermis to cause the pooled region to form, the pooled region of fluid thus formed can be referred to as a suction blister. For example, pressures of at least about 50 mmHg, at least about 100 mmHg, at least about 150 mmHg, at least about 200 mmHg, at least about 250 mmHg, at least about 300 mmHg, at least about 350 mmHg, at least about 400 mmHg, at least about 450 mmHg, at least about 500 mmHg, at least about 550 mmHg, at least about 600 mmHg, at least about 650 mmHg, at least about 700 mmHg, or at least about 750 mmHg may be applied to the skin, e.g., to cause a suction blister and/or to collect interstitial fluid from a subject (as discussed, these measurements are negative relative to atmospheric pressure). For instance, a vacuum pressure of 100 mmHg corresponds to an absolute pressure of about 660 mmHg (i.e., 100 mmHg below 1 atm). Different amounts of vacuum may be applied to different subjects in some cases, for example, due to differences in the physical characteristics of the skin of the subjects.

The vacuum may be applied to any suitable region of the skin, and the area of the skin to which the vacuum may be controlled in some cases. For instance, the average diameter of the region to which vacuum is applied may be kept to less than about 5 cm, less than about 4 cm, less than about 3 cm, less than about 2 cm, less than about 1 cm, less than about 5 mm, less than about 4 mm, less than about 3 mm, less than about 2 mm, or less than about 1 mm In addition, such vacuums may be applied for any suitable length of time at least sufficient to cause at least some separation of the dermis from the epidermis to occur. For instance, vacuum may be applied to the skin for at least about 1 min, at least about 3 min, at least about 5 min, at least about 10 min, at least about 15 min, at least about 30 min, at least about 1 hour, at least about 2 hours, at least about 3 hours, at least 4 hours, etc. Examples of devices suitable for creating such suction blisters are discussed in more detail below. In other cases, however, bodily fluids such as blood or interstitial fluid may be removed from the skin using vacuum without the creation of a suction blister. Other non-limiting fluids include saliva, sweat, tears, mucus, plasma, lymph, or the like.

In one set of embodiments, a pressure differential (e.g. a vacuum) may be created by a pressure regulator. As used here, “pressure regulator” is a pressure controller component or system able to create a pressure differential between two or more locations. The pressure differential should be at least sufficient to urge the movement of fluid or other material in accordance with various embodiments of the invention as discussed herein, and the absolute pressures at the two or more locations are not important so long as their differential is appropriate, and their absolute values are reasonable for the purposes discussed herein. For example, the pressure regulator may produce a pressure higher than atmospheric pressure in one location, relative to a lower pressure at another location (atmospheric pressure or some other pressure), where the differential between the pressures is sufficient to urge fluid in accordance with the invention. In another example, the regulator or controller will involve a pressure lower than atmospheric pressure (a vacuum) in one location, and a higher pressure at another location(s) (atmospheric pressure or a different pressure) where the differential between the pressures is sufficient to urge fluid in accordance with the invention. Wherever “vacuum” or “pressure” is used herein, in association with a pressure regulator or pressure differential of the invention, it should be understood that the opposite can be implemented as well, as would be understood by those of ordinary skill in the art, i.e., a vacuum chamber can be replaced in many instances with a pressure chamber, for creating a pressure differential suitable for urging the movement of fluid or other material.

The pressure regulator may be an external source of vacuum (e.g. a lab, clinic, hospital, etc., house vacuum line or external vacuum pump), a mechanical device, a vacuum chamber, pre-packaged vacuum chamber, or the like. Vacuum chambers can be used in some embodiments, where the device contains, e.g., regions in which a vacuum exits or can be created (e.g. a variable volume chamber, a change in volume of which will affect vacuum or pressure). A vacuum chamber can include pre-evacuated (i.e., pre-packaged) chambers or regions, and/or self-contained actuators.

A “self-contained” vacuum (or pressure) regulator means one that is associated with (e.g., on or within) the device, e.g. one that defines an integral part of the device, or is a separate component constructed and arranged to be specifically connectable to the particular device to form a pressure differential (i.e., not a connection to an external source of vacuum such as a hospital's, clinic's, or lab's house vacuum line, or a vacuum pump suitable for very general use). In some embodiments, the self-contained vacuum source may be actuated in some fashion to create a vacuum within the device. For instance, the self-contained vacuum source may include a piston, a syringe, a mechanical device such as a vacuum pump able to create a vacuum within the device, and/or chemicals or other reactants that can react to increase or decrease pressure which, with the assistance of mechanical or other means driven by the reaction, can form a pressure differential associated with a pressure regulator. Chemical reaction can also drive mechanical actuation with or without a change in pressure based on the chemical reaction itself. A self-contained vacuum source can also include an expandable foam, a shape memory material, or the like.

One category of self-contained vacuum or pressure regulators of the invention includes self-contained assisted regulators. These are regulators that, upon actuation (e.g., the push of a button, or automatic actuation upon, e.g., removal from a package or urging a device against the skin), a vacuum or pressure associated with the device is formed where the force that pressurizes or evacuates a chamber is not the same as the actuation force. Examples of self-contained assisted regulators include chambers evacuated by expansion driven by a spring triggered by actuation, release of a shape-memory material or expandable material upon actuation, initiation of a chemical reaction upon actuation, or the like.

Another category of self-contained vacuum or pressure regulators of the invention are devices that are not necessarily pre-packaged with pressure or vacuum, but which can be pressurized or evacuated, e.g. by a subject, health care professional at a hospital or clinic prior to use, e.g. by connecting a chamber of the device to a source of vacuum or pressure. For example, the subject, or another person, may actuate the device to create a pressure or vacuum within the device, for example, immediately prior to use of the device.

The vacuum or pressure regulator may be a “pre-packaged” pressure or vacuum chamber in the device when used (i.e., the device can be provided ready for use by a subject or practitioner with an evacuated region on or in the device, without the need for any actuation to form the initial vacuum). A pre-packaged pressure or vacuum chamber regulator can, e.g., be a region evacuated (relative to atmospheric pressure) upon manufacture and/or at some point prior to the point at which it is used by a subject or practitioner. For example, a chamber is evacuated upon manufacture, or after manufacture but before delivery of the device to the user, e.g. the clinician or subject. For instance, in some embodiments, the device contains a vacuum chamber having a vacuum of at least about 50 mmHg, at least about 100 mmHg, at least about 150 mmHg, at least about 200 mmHg, at least about 250 mmHg, at least about 300 mmHg, at least about 350 mmHg, at least about 400 mmHg, at least about 450 mmHg, at least about 500 mmHg, at least about 550 mmHg, at least about 600 mmHg, at least about 650 mmHg, at least about 700 mmHg, or at least about 750 mmHg below atmospheric pressure. However, other methods besides vacuum may be used to cause such separation to occur. For example, in another set of embodiments, heat may be used. For instance, a portion of the skin may be heated to at least about 40° C., at least about 50° C., or at least about 55° C., using any suitable technique, to cause such separation to occur. In some (but not all) cases, the temperature may be limited to no more than about 60° C. or no more than about 55° C. The skin may be heated, for instance, using an external heat source (e.g., radiant heat or a heated water bath), a chemical reaction, electromagnetic radiation (e.g., microwave radiation, infrared radiation, etc.), or the like. In some cases, the radiation may be focused on a relatively small region of the skin, e.g., to at least partially spatially contain the amount of heating within the skin that occurs.

In yet another set of embodiments, a separation chemical may be applied to the skin to at least partially cause separation of the dermis and the epidermis to occur. Non-limiting examples of such separation chemicals include proteases such as trypsin, purified human skin tryptase, or compound 48/80. Separation compounds such as these are commercially available from various sources. The separation chemical may be applied directly to the skin, e.g., rubbed into the surface of the skin, or in some cases, the separation chemical can be delivered into the subject, for example, between the epidermis and dermis of the skin. The separation chemical can, for example, be injected in between the dermis and the epidermis.

Another example of a separation chemical is a blistering agent, such as pit viper venom or blister beetle venom. Non-limiting examples of blistering agents include phosgene oxime, Lewisite, sulfur mustards (e.g., mustard gas or 1,5-dichloro-3-thiapentane, 1,2-bis(2-chloroethylthio)ethane, 1,3-bis(2-chloroethylthio)-n-propane, 1,4-bis(2-chloroethylthio)-n-butane, 1,5-bis(2-chloroethylthio)-n-pentane, 2-chloroethylchloromethylsulfide, bis(2-chloroethyl)sulfide, bis(2-chloroethylthio)methane, bis(2-chloroethylthiomethyl)ether, or bis(2-chloroethylthioethyl)ether), or nitrogen mustards (e.g., bis(2-chloroethyl)ethylamine, bis(2-chloroethyl)methylamine, or tris(2-chloroethyl)amine).

In still another set of embodiments, a device may be inserted into the skin and used to mechanically separate the epidermis and the dermis, for example, a wedge or a spike. Fluids may also be used to separate the epidermis and the dermis, in yet another set of embodiments. For example, saline or another relatively inert fluid may be injected into the skin between the epidermis and the dermis to cause them to at least partially separate. These and/or other techniques may also be combined, in still other embodiments. For example, in one embodiment, vacuum pressure and heat may be applied to the skin of a subject, sequentially and/or simultaneously, to cause such separation to occur. As a specific example, in one embodiment, vacuum is applied while the skin is heated to a temperature of between about 40° C. and about 50° C.

In certain embodiments, the skin delivery device is able to create a pooled region of fluid within the skin of a subject. In one embodiment, the device is able to create vacuum pressure on the surface of the skin, e.g., to form a suction blister within the skin. For example, vacuum pressures of at least about 50 mmHg, at least about 100 mmHg, at least about 150 mmHg, at least about 200 mmHg, at least about 250 mmHg, or at least about 300 mmHg may be applied to the skin to cause a suction blister. Any source of vacuum pressure may be used. For example, the device may comprise a vacuum pressure source, and/or be connectable to a vacuum pressure source is external to the device, such as a vacuum pump or an external (line) vacuum source. In some cases, vacuum pressure may be created manually, e.g., by manipulating a syringe pump or the like, or the low pressure may be created mechanically or automatically, e.g., using a piston pump or the like.

Thus, certain embodiments of the invention are directed to causing the formation of a pooled region of fluid between the dermis and epidermis, such as in a suction blister. Fluid may be removed from the pooled region and analyzed in some fashion, e.g., to determine oxygen, or material (e.g., particles) may be delivered to the pooled region of fluid. For example, in one set of embodiments, various particles may be delivered to the fluid, whether pooled between the dermis or epidermis, or in fluid removed from the subject, and the particles can be used to determine oxygen. Optionally, fluid within a pooled region may be drained, e.g., externally, or the fluid may be resorbed, which may leave particles or other material embedded within the skin between the epidermis and dermis.

In one set of embodiments, a pooled region of fluid can be created between the dermis and epidermis of the skin. Suction blisters or other pooled regions may form in a manner such that the suction blister or other pooled region is not significantly pigmented in some cases, since the basal layer of the epidermis contains melanocytes, which are responsible for producing pigments. Such regions can be created by causing the dermis and the epidermis to at least partially separate, and a number of techniques can be used to at least partially separate the dermis from the epidermis.

In one technique, a pool of fluid is formed between layers of skin of a subject and, after forming the pool, fluid is drawn from the pool by accessing the fluid through a layer of skin, for example, puncturing the outer layer of skin with a microneedle. Specifically, for example, a suction blister can be formed and then the suction blister can be punctured and fluid can be drawn from the blister. In another technique, an interstitial region can be accessed and fluid drawn from that region without first forming a pool of fluid via a suction blister or the like. For example, a microneedle or microneedles can be applied to the interstitial region and fluid can be drawn there from. Where microneedles are used, it can be advantageous to select needles of length such that interstitial fluid is preferentially obtained and, where not desirable, blood is not accessed. Those of ordinary skill in the art can arrange microneedles relative to the skin for these purposes including, in one embodiment, introducing microneedles into the skin at an angle, relative to the skin's surface, other than 90°, i.e., to introduce a needle or needles into the skin in a slanting fashion so as to access interstitial fluid.

Another example of a separation chemical is a blistering agent, such as pit viper venom or blister beetle venom. Non-limiting examples of blistering agents include phosgene oxime, Lewisite, sulfur mustards (e.g., mustard gas or 1,5-dichloro-3-thiapentane, 1,2-bis(2-chloroethylthio)ethane, 1,3-bis(2-chloroethylthio)-n-propane, 1,4-bis(2-chloroethylthio)-n-butane, 1,5-bis(2-chloroethylthio)-n-pentane, 2-chloroethylchloromethylsulfide, bis(2-chloroethyl)sulfide, bis(2-chloroethylthio)methane, bis(2-chloroethylthiomethyl)ether, or bis(2-chloroethylthioethyl)ether), or nitrogen mustards (e.g., bis(2-chloroethyl)ethylamine, bis(2-chloroethyl)methylamine, or tris(2-chloroethyl)amine).

The fluid contained within the skin, e.g., within the pooled region of fluid is typically drawn from the surrounding dermal and/or epidermal layers within the skin, and includes interstitial fluid, or even blood in some cases. In some cases, such fluids may be collected even without creating a suction blister within the skin. For instance, a vacuum may be applied to the skin, e.g., through a needle as described herein, to withdraw interstitial fluid from the skin.

In some embodiments, the present invention is generally directed to devices able to cause the formation of the pooled region of fluids within the skin of a subject, and in some cases, to devices able to deliver and/or remove fluids or other materials from the pooled region of fluids. It should be understood, however, that other devices in other aspects do not require the formation of pooled regions of fluids within the skin In some cases, the device may be able to collect bodily fluids such as interstitial fluid or blood from the skin, including fluid from a pooled region of fluid, or from other locations. For example, the device may take the form of a skin “patch,” according to one embodiment. Typically, a skin patch includes one or more layers of material that are adhered to the surface of the skin, and can be applied by the subject or another person. In certain embodiments, layers or portions of the skin patch may be removed, leaving other layers or portions behind on the skin. Often, the skin patch lacks an external power source, although the various layers of the patch may contain various chemicals, such as drugs, therapeutic agents, diagnostic agents, reaction entities, etc. In some cases, the skin patch may also include mechanical elements as well, for example, a cutter such as is discussed herein.

As a specific, non-limiting example, in one embodiment, a skin patch or other device may be used to create a suction blister without an external power and/or a vacuum source. Examples of such devices include, besides skin patches, strips, tapes, bandages, or the like. For instance, a skin patch may be contacted with the skin of a subject, and a vacuum created through a change in shape of a portion of the skin patch or other device (e.g., using a shape memory polymer), which may be used to create a suction blister and/or withdraw fluid from the skin As a specific example, a shape memory polymer may be shaped to be flat at a first temperature (e.g., room temperature) but curved at a second temperature (e.g., body temperature), and when applied to the skin, the shape memory polymer may alter from a flat shape to a curved shape, thereby creating a vacuum. As another example, a mechanical device may be used to create the vacuum, For example, springs, coils, expanding foam (e.g., from a compressed state), a shape memory polymer, shape memory metal, or the like may be stored in a compressed or wound released upon application to a subject, then released (e.g., unwinding, uncompressing, etc.), to mechanically create the vacuum. Thus, in some cases, the device is “pre-packaged” with a suitable vacuum source (e.g., a pre-evacuated vacuum chamber); for instance, in one embodiment, the device may be applied to the skin and activated in some fashion to create and/or access the vacuum source. In yet another example, a chemical reaction may be used to create a vacuum, e.g., a reaction in which a gas is produced, which can be harnessed to provide the mechanical force to create a vacuum. In still another example, a component of the device may be able to create a vacuum in the absence of mechanical force. In another example, the device may include a self-contained vacuum actuator, for example, chemical reactants, a deformable structure, a spring, a piston, etc.

In certain embodiments, the device is able to create a pooled region of fluid within the skin of a subject. In one embodiment, the device is able to create vacuum on the surface of the skin, e.g., to form a suction blister within the skin. In other embodiments, however, the device may create a vacuum to withdraw fluid from the skin without necessarily creating a pooled region of fluid or forming a suction blister within the skin In one set of embodiments, fluids may be delivered to or withdrawn from the skin using vacuum. The vacuum may be an external vacuum source, and/or the vacuum source may be self-contained within the device. For example, vacuums of at least about 50 mmHg, at least about 100 mmHg, at least about 150 mmHg, at least about 200 mmHg, at least about 250 mmHg, at least about 300 mmHg, at least about 350 mmHg, at least about 400 mmHg, at least about 450 mmHg, at least about 500 mmHg, at least 550 mmHg, at least 600 mmHg, at least 650 mmHg, at least about 700 mmHg, or at least about 750 mmHg may be applied to the skin to cause a suction blister. Any source of vacuum may be used. For example, the device may comprise a vacuum source, and/or be connectable to a vacuum source is external to the device, such as a vacuum pump or an external (line) vacuum source. In some cases, vacuum may be created manually, e.g., by manipulating a syringe pump or the like, or the low pressure may be created mechanically or automatically, e.g., using a piston pump, a syringe, a bulb, a Venturi tube, manual (mouth) suction, etc. or the like.

As mentioned, any source of vacuum may be used. For example, the device may comprise an internal vacuum source, and/or be connectable to a vacuum source is external to the device, such as a vacuum pump or an external (line) vacuum source.

The device may also comprise, in some cases, a portion able to deliver materials such as particles into the pooled region within the skin. For example, the device may include a needle such as a hypodermic needle or microneedles, or jet injectors such as those discussed herein.

In one set of embodiments, a device of the present invention may not have an external power and/or a vacuum source. In some cases, the device is “pre-loaded” with a suitable vacuum source; for instance, in one embodiment, the device may be applied to the skin and activated in some fashion to create and/or access the vacuum source. As one example, a device of the present invention may be contacted with the skin of a subject, and a vacuum created through a change in shape of a portion of the device (e.g., using a shape memory polymer), or the device may contain one or more sealed, self-contained vacuum compartments, where a seal is punctured in some manner to create a vacuum. For instance, upon puncturing the seal, a vacuum compartment may be in fluidic communication with a needle, which can be used to move the skin towards the device, withdraw fluid from the skin, or the like.

In one set of embodiments, the device may include a sensor, for example embedded within or integrally connected to the device, or positioned remotely but with physical, electrical, and/or optical connection with the device so as to be able to sense a compartment within the device. For example, the sensor may be in fluidic communication with fluid withdrawn from a subject, directly, via a microfluidic channel, an analytical chamber, etc. The sensor may be able to sense an analyte, e.g., oxygen. For example, a sensor may be free of any physical connection with the device, but may be positioned so as to detect the results of interaction of electromagnetic radiation, such as infrared, ultraviolet, or visible light, which has been directed toward a portion of the device, e.g., a compartment within the device. As another example, a sensor may be positioned on or within the device, and may sense activity in a compartment by being connected optically to the compartment. Sensing communication can also be provided where the compartment is in communication with a sensor fluidly, optically or visually, thermally, pneumatically, electronically, or the like, so as to be able to sense a condition of the compartment. As one example, the sensor may be positioned downstream of a compartment, within a channel such a microfluidic channel, or the like.

In some embodiments, signaling methods such as these may be used to indicate the presence and/or concentration of an analyte determined by the sensor, e.g., to the subject, and/or to another entity, such as those described below. Where a visual signal is provided, it can be provided in the form of change in opaqueness, a change in intensity of color and/or opaqueness, or can be in the form of a message (e.g., numerical signal, or the like), an icon (e.g., signaling by shape or otherwise a particular medical condition), a brand, logo, or the like. For instance, in one embodiment, the device may include a display. A written message such as “take next dose,” or “glucose level is high” or a numerical value might be provided, or a message such as “toxin is present.” These messages, icons, logos, or the like can be provided as an electronic read-out by a component of a device and/or can be displayed as in inherent arrangement of one or more components of the device.

In connection with any signals associated with any analyses described herein, another, potentially related signal or other display (or smell, taste, or the like) can be provided which can assist in interpreting and/or evaluating the signal. In one arrangement, a calibration or control is provided proximate to (or otherwise easily comparable with) a signal, e.g., a visual calibration/control or comparator next to or close to a visual signal provided by a device and/or implanted agents, particles, or the like.

In one embodiment, the device is able to transmit a signal to another entity. The entity that the signal is transmitted to can be a human (e.g., a clinician) or a machine. In some cases, the other entity may be able to analyze the signal and take appropriate action. In one arrangement, the other entity is a machine or processor that analyzes the signal and optionally sends a signal back to the device to give direction as to activity (e.g., a cell phone can be used to transmit an image of a signal to a processor which, under one set of conditions, transmits a signal back to the same cell phone giving direction to the user, or takes other action). Other actions can include automatic stimulation of the device or a related device to dispense a medicament or pharmaceutical, or the like. The signal to direct dispensing of a pharmaceutical can take place via the same used to transmit the signal to the entity (e.g., cell phone) or a different vehicle or pathway. Telephone transmission lines, wireless networks, Internet communication, and the like can also facilitate communication of this type.

Information regarding the analysis can also be transmitted to the same or a different entity, or a different location simply by removing the device or a portion of the device from the subject and transferring it to a different location. For example, a device can be used in connection with a subject to analyze presence and/or amount of a particular analyte. At some point after the onset of use, the device, or a portion of the device carrying a signal or signals indicative of the analysis or analyses, can be removed and, e.g., attached to a record associated with the subject. As a specific example, a patch or other device can be worn by a subject to determine presence and/or amount of one or more analytes qualitatively, quantitatively, and/or over time. The subject can visit a clinician who can remove the patch (or other device) or a portion of the patch and in some cases, attach it to a medical record associated with the subject.

In some aspects, the particles may be subsequently removed from the skin. As mentioned, in one set of embodiments, the particles may be present in the epidermis and slough off with the epidermis naturally, e.g., on the time scale of days to weeks, depending on the depth of penetration. In other embodiments, however, an externally applied stimulus is applied to the skin of the subject to at least partially remove and/or inactivate the particles. For instance, light, such as laser light, may be applied to the skin to ablate at least a portion of the skin, including the particles. In some cases, however, light may be applied to inactivate a portion of the particles (e.g., a reaction entity on the surface of the particles). Many skin ablation lasers may be obtained commercially (for instance, an Er:YAG-laser or a carbon dioxide laser), which are used, for instance, for laser skin resurfacing, facial rejuvenation, ablative removal of skin lesions, or the like. Ablation rates in the skin can be controlled, for instance, by controlling the fluence rate of the laser, the number and/or frequency of pulses (in a pulsed laser), or the like.

As mentioned, although the discussions above describe embodiments in which the concentration of oxygen within a subject is determined (e.g., within the skin, or within other organs within the subject), this is by way of example only, and in other cases, oxygen concentrations in other samples, may also be determined. For example, in one set of embodiments, particles or other agents that exhibit a determinable change when exposed to different concentrations or amounts of oxygen may be administered to a tissue or an isolated organ, for instance, to monitor the oxygen concentration within such tissues or organs. As a specific example, if the organ is one that is being transplanted, such particles may be used to monitor the condition of the organ during the transplant procedure (e.g., during transport of the organ from one location to another).

The present invention may also find use in various applications where it is desired to determine oxygen concentrations or amounts in subjects having certain health conditions, for instance, having or at risk for sleep apnea, pressure ulcers or blisters, bed sores, or in certain infants. For instance, in one set of embodiments, the oxygen concentration within the blood or within the skin (or portion thereof) of a subject may be determined by administering particles or other suitable agents to the subject. In some cases, a specific location, e.g., region of the skin of the subject, may be determined to determine oxygen concentrations or amounts. The particles may be administered using a skin patch or other skin delivery device, as discussed above, injected into the subject, or the like.

As a specific example, in one embodiment of the invention, a subject suspected of having sleep apnea is exposed to such particles or other agents, e.g., prior to sleeping. Sleep apnea is a sleep disorder generally characterized by pauses in breathing during sleep, which can cause low levels of oxygen within the subject due to the lack of breathing. During or after sleeping, the administered particles can then be determined to determine whether the particles have been exposed to relatively low concentrations of oxygen, which may indicate that the subject has sleep apnea. In one embodiment, particles that aggregate irreversibly when exposed to relatively low oxygen concentrations may be used, e.g., particles at least partially coated with certain forms of hemoglobin, such as certain sickle-cell hemoglobins. Thus, even after the subject subsequently resumes breathing, or upon awakening, the low levels of oxygen may still be determined, and used to diagnose the condition of the subject.

As another example, particles or other agents of the invention may be present as part of an article that can placed on the head of a baby prior to birth, but after crowning, in order to monitor the amount of oxygen present within the baby's blood, e.g., during the birthing process.

Additional non-limiting examples of various embodiments of the invention for determining oxygen in a sample, or in a subject will now be described. In addition, in some cases, these may be combined with any of the embodiments previously described.

For instance, one aspect is directed to using one or more reactive agents to detect oxygen, e.g., within a device. In some cases, oxygen and another analyte in addition to oxygen may be determined. Other analytes in addition to oxygen may also be determined in some cases. In some embodiments, a reactive agent may react with the analyte to be detected or measured and the second creates the detectable signal, e.g., visual and/or tactile signals—smell, taste, shape change, or the like. Combinations of each can be utilized. For example, antibody to carcinoembryonic antigen (“CEA”) and antibody to prostate specific antigen (“PSA”) may be used to monitor for cancer of either origin; colors may be yellow for CEA and blue for PSA, resulting in green if both are elevated.

The term “reactive partner” refers to a molecule that can undergo binding or reaction with a particular molecule, e.g., an analyte. “Binding partners” are defined herein as any molecular species which form highly specific, non-covalent, physiochemical interactions with each other. Binding partners which form highly specific, non-covalent, physiochemical interactions with one another are defined herein as “complementary.” Many suitable binding partners are known in the art, and include any molecular species, including, but not limited to antibody/antigen pairs, ligand/receptor pairs, enzyme/substrate pairs and complementary nucleic acids or aptamers. Examples of suitable epitopes which may be used for antibody/antigen binding pairs include, but are not limited to, HA, FLAG, c-Myc, glutatione-S-transferase, His₆, GFP, DIG, biotin and avidin. Antibodies which bind to these epitopes are well known in the art. Antibodies may be monoclonal or polyclonal. Suitable antibodies for use as binding partners include antigen-binding fragments, including separate heavy chains, light chains Fab, Fab′ F(ab′)2, Fabc, and Fv. Antibodies also include bispecific or bifunctional antibodies. Exemplary binding partners include biotin/avidin, biotin/streptavidin, biotin/neutravidin and glutathione-S-transferase/glutathione.

For example, Protein A is a reactive partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Reactive partners include specific, semi-specific, and non-specific reactive partners. Protein A is usually regarded as a “non-specific” or semi-specific binder. An enzyme such as glucose oxidase or glucose 1-dehydrogenase, or a lectin such as concanavalin A that is able to bind to glucose, may also be utilized.

The term “binding” generally refers to the interaction between a corresponding pair of molecules or surfaces that exhibit mutual affinity or binding capacity, typically due to specific or non-specific binding or interaction, including, but not limited to, biochemical, physiological, and/or chemical interactions. The binding may be between biological molecules, including proteins, nucleic acids, glycoproteins, carbohydrates, hormones, or the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc. As another example, the binding agent may be a chelating agent (e.g., ethylenediaminetetraacetic acid) or an ion selective polymer (e.g., a block copolymer such as poly(carbonate-b-dimethylsiloxane), a crown ether, or the like). In some cases, binding may be between non-biological molecules, for example, between a catalyst and its substrate. The reactive partners and the species may be biotin and streptavidin, or the reactive partners may be various antibodies raised against a protein.

The term “specifically binds,” when referring to a reactive partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair, the ligand would specifically and/or preferentially select its receptor from a complex mixture of molecules, or vice versa. An enzyme would specifically bind to its substrate, a nucleic acid would specifically bind to its complement, an antibody would specifically bind to its antigen, etc. The binding may be by one or more of a variety of mechanisms including, but not limited to ionic interactions or electrostatic interactions, covalent interactions, hydrophobic interactions, van der Waals interactions, etc.

As an example, an analyte may cause a determinable change in a property of the particles, e.g., a change in a chemical property of the particles, a change in the appearance and/or optical properties of the particles, a change in the temperature of the particles, a change in an electrical property of the particles, etc. In some cases, the change may be one that is determinable by a human, unaided by any equipment that may be directly applied to the human. For instance, the determinable change may be a change in appearance (e.g., color), a change in temperature, the production of an odor, etc., which can be determined by the human eye without the use of any equipment.

Reactive partners to these and/or other species are well-known in the art. Non-limiting examples include pH-sensitive entities such as phenol red, bromothymol blue, chlorophenol red, fluorescein, HPTS, 5(6)-carboxy-2′,7′-dimethoxyfluorescein SNARF, and phenothalein; entities sensitive to calcium such as Fura-2 and Indo-1; entities sensitive to chloride such as 6-methoxy-N-(3-sulfopropyl)-quinolinim and lucigenin; entities sensitive to nitric oxide such as 4-amino-5-methylamino-2′,7′-difluorofluorescein; entities sensitive to dissolved oxygen such as tris(4,4′-diphenyl-2,2′-bipyridine) ruthenium (II) chloride pentahydrate; entities sensitive to dissolved CO2; entities sensitive to fatty acids, such as BODIPY 530-labeled glycerophosphoethanolamine; entities sensitive to proteins such as 4-amino-4′-benzamidostilbene-2-2′-disulfonic acid (sensitive to serum albumin), X-Gal or NBT/BCIP (sensitive to certain enzymes), Tb3+ from TbCl3 (sensitive to certain calcium-binding proteins), BODIPY FL phallacidin (sensitive to actin), or BOCILLIN FL (sensitive to certain penicillin-binding proteins); entities sensitive to concentration of glucose, lactose or other components, or entities sensitive to proteases, lactates or other metabolic byproducts, entities sensitive to proteins, antibodies, or other cellular products.

Other properties besides color may be determined, e.g., temperature changes, chemical reactions (e.g., capsaicin). Examples of capsaicin and capsaicin-like molecules include, but are not limited to, dihydrocapsaicin, nordihydrocapsaicin, homodihydrocapsaicin, homocapsaicin, or nonivamide.

More than one analyte may be determined, e.g., oxygen and another analyte. For instance, a first set of particles and/or reactive agents may determine a first analyte and a second set of particles and/or reactive agents may determine a second analyte. This may be used to determine a physical condition of a subject. For instance, a first color may indicate a healthy state and a second color indicate a disease state. In some cases, the appearance may be used to determine a degree of health. For instance, a first color may indicate a healthy state, a second color may indicate a warning state, and a third may indicate a dangerous state, or a range of colors indicate a degree of health of the subject.

The reactive agents may be colored or react to produce color, shape change (for example, if the polymer is a shape memory polymer or a “smart polymer” to produce or release color or another indicator, hydrolyse or release when reacted, or aggregate to intensify when reacted. As a specific example, the first set of reactive agents may be colored yellow and blue, and the second set of reactive agents may be colored red and blue. If no analyte is present, the reactive agents are randomly oriented, giving a dark appearance (i.e., red+yellow+blue). If the analyte is present, but at low concentrations, the first set of reactive agents may be able to bind the analyte but not the second set of reactive agents, as the first set of reactive agents contain a higher concentration of reactive agents able to recognize the analyte. Thus the first set of reactive agents may exhibit more yellow than blue (e.g., due to aggregation of the first set of reactive agents to the analyte; the first set of reactive agents may aggregate around the analyte to a greater degree than the second set of reactive agents), and the overall appearance of the reactive agents shifts to a dark yellow appearance. At higher concentrations of analyte, both sets of reactive agents may be able to bind the analyte, and the second set of reactive agents may exhibit more red than blue (e.g., due to aggregation of the second set of reactive agents). The overall appearance of the reactive agents may then shift to an orange appearance (red+yellow).

Alternatively, an optical property of the medium containing the clusters may be altered in some fashion (e.g., exhibiting different light scattering properties, different opacities, different degrees of transparency, etc.), which can be correlated with the analyte. In some cases, the color may change in intensity, for example, the clustering of particles may bring two or more reactants into close proximity.

The reactants may react in some fashion that can be determined, e.g., by producing light, producing heat, pH change, release of gas, smell, taste, etc. In some cases, a precipitate and/or flocculate may be formed—or may disperse. In another example, clustering of reactive agents may cause a change in an electrical or a magnetic property of the reactive agents, which can be indicative of a change in an electrical or a magnetic field. This can also be achieved or altered by use of an external electrical, magnetic, and/or a mechanical force.

The reaction between the first and second reactive agents may be an endothermic or an exothermic reaction; resulting in a detectable temperature change. As an example, the first reactive agent may contain barium hydroxide (Ba(OH)₂), while the second reactive agent may contain ammonium nitrate (NH₄NO₃). The reactive agent may be present in solution or suspension, and only a low level of reaction between the barium hydroxide and the ammonium nitrate occurs. However, when a species is added which is recognized by the reactive partners on the first and second reactive agent, aggregation of the reactive agents may occur. As the reactive agents aggregate such that the first halves orient on the species, the second halves may also be brought into closer proximity, allowing the reaction rate between the reactants to increase. In this case, the reaction between barium hydroxide and the ammonium nitrate is an endothermic reaction that yields barium nitrate (Ba(NO₃)₂) and ammonium (NH₃). This may be determined by determining a drop in temperature. The first half of the reactive agents also contains a glucose reactive partner, such as a lectin (e.g., concanavalin A), glucose oxidase or glucose 1-dehydrogenase that is able to bind to glucose. At relatively low levels of glucose, no aggregation of the reactive agents occurs, and no change in temperature is felt by the subject. However, at relatively high levels of glucose, some aggregation of the reactive agents occurs, such that the particles orient around the glucose, where the first halves of the reactive agents orients to the glucose due to the presence of the glucose reactive partner. The second halves of the reactive agents are thus brought into close proximity to each other, allowing the reaction rate between the reactants to increase. In this case, the reaction between barium hydroxide and the ammonium nitrate is an endothermic reaction that yields barium nitrate (Ba(NO₃)₂) and ammonium (NH₃). This may be sensed as a drop in temperature.

Irritation or pain can also be used as the signal that is detected. For example, a glucose sensor can be prepare from particles formed of a biocompatible polymer such as PEO, or a polymer of polylactic acid and/or polyglycolic acid. The first set of anisotropic particles contains a first half containing a reactive partner to a species and a second half that contains a first reactant, while the second set of anisotropic particles also contains a reactive partner to the species (which may be the same or different than the reactive partner of the first set of particles) and a second half that contains a second reactant. The first and second reactants may be, for example, two reactants that cause the release of capsaicin or a capsaicin-like molecule such as dihydrocapsaicin, which may be felt by a subject as pain. In one embodiment, the first reactant may be a liposome that contains the capsaicin or capsaicin-like molecule and the second reactant may be a lipase able to degrade the liposome, thereby releasing the capsaicin from the liposome. The first half of the particles also contains a glucose reactive partner, such as a lectin (e.g., concanavalin A), glucose oxidase or glucose 1-dehydrogenase that is able to bind to glucose.

In another embodiment, the binding or presence of the analyte results in a tactile change (e.g., change in shape or texture) in the composition. For example, shape memory polymer (SMPs) can be used to detect the presence of one or more analytes.

SMPs may be characterized as phase segregated linear block co-polymers having a hard segment and a soft segment. The hard segment is typically crystalline, with a defined melting point, and the soft segment is typically amorphous, with a defined glass transition temperature. In some embodiments, however, the hard segment is amorphous and has a glass transition temperature rather than a melting point. In other embodiments, the soft segment is crystalline and has a melting point rather than a glass transition temperature. The melting point or glass transition temperature of the soft segment is substantially less than the melting point or glass transition temperature of the hard segment.

When the SMP is heated above the melting point or glass transition temperature of the hard segment, the material can be shaped, according to some embodiments. This (original) shape can be memorized by cooling the SMP below the melting point or glass transition temperature of the hard segment. When the shaped SMP is cooled below the melting point or glass transition temperature of the soft segment while the shape is deformed, that (temporary) shape may be fixed in some cases. The original shape can be recovered by heating the material above the melting point or glass transition temperature of the soft segment but below the melting point or glass transition temperature of the hard segment. The recovery of the original shape, which may be induced by an increase in temperature, is called the thermal shape memory effect. Properties that describe the shape memory capabilities of a material include the shape recovery of the original shape and the shape fixity of the temporary shape.

Shape memory polymers can contain at least one physical crosslink (physical interaction of the hard segment) or contain covalent crosslinks instead of a hard segment. The shape memory polymers also can be interpenetrating networks or semi-interpenetrating networks. In addition to changes in state from a solid to liquid state (melting point or glass transition temperature), hard and soft segments may undergo solid to solid state transitions, and can undergo ionic interactions involving polyelectrolyte segments or supramolecular effects based on highly organized hydrogen bonds.

Other polymers that can shape or phase change as a function of temperature include PLURONICS®. These are also known as poloxamers, nonionic triblock copolymers composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist that have slightly different properties. For the generic term “poloxamer,” these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the PLURONICS® tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). In the example given, poloxamer 181 (P181)=Pluronic L61. PLURONICS® are described in U.S. Pat. No. 3,740,421.

Other temperature sensitive polymers that form gels that have a distinct phase change at its lower critical solution temperature (LCST) including the cross-linked copolymers comprising hydrophobic monomers, hydrogen bonding monomers, and thermosensitive monomers described in U.S. Pat. No. 6,538,089 to Samra, et al.

Additional thermal responsive, water soluble polymers including the co-polymerization product of N-isopropyl acrylamide (NIP); 1-vinyl-2-pyrrolidinone (VPD); and optionally, acrylic acid (AA), change shape as a function of temperature. As the proportion of component AA increases, the Lower Critical Solution Temperature (LCST) decreases and the COOH reactive groups increase, which impart high reactivity to the copolymer. By adjusting the proportion of the monomers, a broad range of LCST can be manipulated from about 20 to 80° C., as described in U.S. Pat. No. 6,765,081 to Lin, et al.

While the shape memory effect is typically described in the context of a thermal effect, the polymers can change their shape in response to application of light, changes in ionic concentration and/or pH, electric field, magnetic field or ultrasound. For example, a SMP can include at least one hard segment and at least one soft segment, wherein at least two of the segments, e.g., two soft segments, are linked to each other via a functional group that may be cleavable under application of light, electric field, magnetic field or ultrasound. The temporary shape may be fixed by crosslinking the linear polymers. By cleaving those links the original shape can be recovered. The stimuli for crosslinking and cleaving these bonds can be the same or different.

In one embodiment, the shape memory polymer composition binds, complexes to, or interacts with an analyte, which can be a chromophore. The hard and/or soft segments can include double bonds that shift from cis to trans isomers when the chromophores absorb light. Light can therefore be used to detect the presence of a chromophore analyte by observing whether or not the double bond isomerizes.

The shape memory effect can also be induced by changes in ionic strength or pH. Various functional groups are known to crosslink in the presence of certain ions or in response to changes in pH. For example, calcium ions are known to crosslink amine and alcohol groups, i.e., the amine groups on alginate can be crosslinked with calcium ions. Also, carboxylate and amine groups become charged species at certain pHs. When these species are charged, they can crosslink with ions of the opposite charge. The presence of groups, which respond to changes in the concentration of an ionic species and/or to changes in pH, on the hard and/or soft segments results in reversible linkages between these segments. One can fix the shape of an object while crosslinking the segments. After the shape has been deformed, alteration of the ionic concentration or pH can result in cleavage of the ionic interactions which formed the crosslinks between the segments, thereby relieving the strain caused by the deformation and thus returning the object to its original shape. Because ionic bonds are made and broken in this process, it can only be performed once. The bonds, however, can be re-formed by altering the ionic concentration and/or pH, so the process can be repeated as desired. Thus, in this embodiment, the presence of an analyte which changes the ionic strength or pH can induce a shape memory effect in the polymer confirming the presence of the analyte.

Electric and/or magnetic fields can also be used to induce a shape memory effect. Various moieties, such as chromophores with a large number of delocalized electrons, increase in temperature in response to pulses of applied electric or magnetic fields as a result of the increased electron flow caused by the fields. After the materials increase in temperature, they can undergo temperature induced shape memory in the same manner as if the materials were heated directly. These compositions are useful in biomedical applications where the direct application of heat to an implanted material may be difficult, but the application of an applied magnetic or electric field would only affect those molecules with the chromophore, and not heat the surrounding tissue. For example, the presence of a chromophore analyte with a large number of delocalized electrons can be cause an increase in temperature in the microenvironment surrounding the shape memory polymer implant in response to pulses of applied electric or magnetic fields. This increase in temperature can in turn cause a thermal shape memory effect, thus confirming the presence of a particular analyte.

Many other types of “smart polymers” are described in U.S. Pat. No. 5,998,588 to Hoffman, et al. The combination of the capabilities of stimuli-responsive components such as polymers and interactive molecules to form site-specific conjugates are useful in a variety of assays, separations, processing, and other uses. The polymer chain conformation and volume can be manipulated through alteration in pH, temperature, light, or other stimuli. The interactive molecules can be biomolecules like proteins or peptides, such as antibodies, receptors, or enzymes, polysaccharides or glycoproteins which specifically bind to ligands, or nucleic acids such as antisense, ribozymes, and aptamers, or ligands for organic or inorganic molecules in the environment or manufacturing processes. The stimuli-responsive polymers are coupled to recognition biomolecules at a specific site so that the polymer can be manipulated by stimulation to alter ligand-biomolecule binding at an adjacent binding site, for example, the biotin binding site of streptavidin, the antigen-binding site of an antibody or the active, substrate-binding site of an enzyme. Binding may be completely blocked (i.e., the conjugate acts as an on-off switch) or partially blocked (i.e., the conjugate acts as a rheostat to partially block binding or to block binding only of larger ligands). Once a ligand is bound, it may also be ejected from the binding site by stimulating one (or more) conjugated polymers to cause ejection of the ligand and whatever is attached to it. Alternatively, selective partitioning, phase separation or precipitation of the polymer-conjugated biomolecule can be achieved through exposure of the stimulus-responsive component to an appropriate environmental stimulus.

Liquid crystal polymeric materials can also be used to provide a signal for detection or quantification of analyte. Liquid crystals are materials that exhibit long-range order in only one or two dimensions, not all three. A distinguishing characteristic of the liquid crystalline state is the tendency of the molecules, or mesogens, to point along a common axis, known as the director. This feature is in contrast to materials where the molecules are in the liquid or amorphous phase, which have no intrinsic order, and molecules in the solid state, which are highly ordered and have little translational freedom. The characteristic orientational order of the liquid crystal state falls between the crystalline and liquid phases. Suitable materials are described in U.S. Patent Application Publication No. 2003/0228367 by Mathiowitz, et al. These can be pressure or temperature sensitive, and react by producing a change in color or shape.

The device optionally includes a carrier which may itself be reactive or which may serve as the “monitor” for the color change. The carrier may be a plastic device which adheres to the surface of the skin or mucosa. It may include a surface containing the reactive agents which change color upon contacting the analyte. It may include agents such as transdermal disrupting agents such as linoleic acid or surfactant to increase transfer of analyte through the skin. It could include mechanical and electrical means, or an ultrasound transducer, to facilitate transfer or to generate a signal, for example, if analyte changes a salt concentration or ion conductivity, or “closes a circuit” to turn on, or off, an LED.

In one embodiment, the reactive agent is also the device. For example, anisotrophic particles can be utilized. Particles having at least two phases or regions may be present on the surfaces of the particles. The particles may be spherical or non-spherical. The particles can be used in a wide variety of applications. For example, the particles may include a reactive partner that when exposed to an analyte recognized by the reactive partner, cause the particles to collect around the analyte, e.g., as an aggregate. The aggregate may produce a visual or other signal distinguishable from the particles in a non-aggregated state, such as a randomly-oriented state. In some cases, the particles, when aggregated, may allow a chemical reaction to occur.

The particles may include microparticles and/or nanoparticles. A “microparticle” is a particle having an average diameter on the order of micrometers (i.e., between about 1 micrometer and about 1 mm), while a “nanoparticle” is a particle having an average diameter on the order of nanometers (i.e., between about 1 nm and about 1 micrometer. The particles may be spherical or non-spherical, in some cases. For example, the particles may be oblong or elongated, or have other shapes such as those disclosed in U.S. patent application Ser. No. 11/851,974, filed Sep. 7, 2007, entitled “Engineering Shape of Polymeric Micro- and Nanoparticles,” by S. Mitragotri, et al.; International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008; U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007, each of which is incorporated herein by reference.

An “anisotropic” particle, as used herein, is one that is not spherically symmetric (although the particle may still exhibit various symmetries). The asymmetry can be asymmetry of shape, of composition, or both. As an example, a particle having the shape of an egg or an American football is not perfectly spherical, and thus exhibits anisotropy. As another example, a sphere painted such that exactly one half is red and one half is blue (or otherwise presents different surface characteristics on different sides) is also anisotropic, as it is not perfectly spherically symmetric, although it would still exhibit at least one axis of symmetry. Accordingly, a particle may be anisotropic due to its shape and/or due to two or more regions that are present on the surface of and/or within the particle. The particle may include a first surface region and a second surface region that is distinct from the first region in some way, e.g., due to coloration, surface coating, the presence of one or more reaction entities, etc. The particle may include different regions only on its surface or the particle may internally include two or more different regions, portions of which extend to the surface of the particle. The regions may have the same or different shapes, and be distributed in any pattern on the surface of the particle. For instance, the regions may divide the particle into two hemispheres, such that each hemisphere has the same shape and/or the same surface area, or the regions may be distributed in more complex arrangements. For instance, a first region may have the shape of a circle on the surface of the particle while the second region occupies the remaining surface of the particle, the first region may be present as a series of distinct regions or “spots” surrounded by the second region, the first and second regions may each be present as a series of “stripes” on the surface of the particle, etc. In some cases, the particle may include three, four, five, or more distinct surface regions. For instance, a particle may include distinct first, second and third surface regions; distinct first, second, third, and fourth surface regions; distinct first, second, third, fourth and fifth surface regions, etc. In some cases, the surface regions may be distinctly colored, and in certain instances, the anisotropic particles may be able to exhibit multiple colors, depending on the external environment. For example, a particle may exhibit a first color in response to a first analyte and a second color in response to a second analyte, as discussed below.

Non-limiting examples of anisotropic particles can be seen in U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007.

U.S. Patent Application Publication No. 2003/0159615 by Anderson, et al., describes a wide variety of microparticles containing and/or formed of colored dyes, which can be used to create a colored signal.

The particles (which may be anisotropic, or not anisotropic) may be formed of any suitable material, depending on the application. For example, the particles may comprise a glass, and/or a polymer such as polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, the particles may comprise a ceramic such as tricalcium phosphate, hydroxyapatite, fluorapatite, aluminum oxide, or zirconium oxide. In some cases (for example, in certain biological applications), the particles may be formed from biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these. In one set of embodiments, the particles may comprise a hydrogel, such as agarose, collagen, or fibrin. The particles may include a magnetically susceptible material in some cases, e.g., a material displaying paramagnetism or ferromagnetism. For instance, the particles may include iron, iron oxide, magnetite, hematite, or some other compound containing iron. In another embodiment, the particles can include a conductive material (e.g., a metal such as titanium, copper, platinum, silver, gold, tantalum, palladium, rhodium, etc.), or a semiconductive material (e.g., silicon, germanium, CdSe, CdS, etc.). Other particles include ZnS, ZnO, TiO₂, AgI, AgBr, HgI₂, PbS, PbSe, ZnTe, CdTe, In₂ S₃, In₂Se₃, Cd₃P₂, Cd₃As₂, InAs, or GaAs. The particles may include other species as well, such as cells, biochemical species such as nucleic acids (e.g., RNA, DNA, PNA, etc.), proteins, peptides, enzymes, nanoparticles, quantum dots, fragrances, indicators, dyes, fluorescent species, chemicals, small molecules (e.g., having a molecular weight of less than about 1 kDa).

In another embodiment, particles, matrices, kits, skin insertion objects, and related species can be those that, based on their degree or amount of dispersion or agglomeration, produce a different signal. For example, certain particles or colloids such as gold nanoparticles can be coated with agents capable of interacting with an analyte. Such particles may associate with each other, or conversely, dissociate in the presence of analyte in such a manner that a change is conferred upon the light absorption property of the material containing the particles. For example, particles coated with complimentary nucleic acid sequences can be used to characterize target nucleic acids complimentary to the particle bound nucleic acids sequence. This approach can also be applied to any class of analyte, in various embodiments, and furthermore can be used as a skin-based visual sensor. A non-limiting example of a technique for identifying aggregates is disclosed in U.S. Pat. No. 6,361,944.

The particles may also have any shape or size. For instance, the particles may have an average diameter of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. As discussed, the particles may be spherical or non-spherical. The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous. Other shapes are also possible, for instance, core/shell structures (e.g., having different compositions), rectangular disks, high aspect ratio rectangular disks, high aspect ratio rods, worms, oblate ellipses, prolate ellipses, elliptical disks, UFOs, circular disks, barrels, bullets, pills, pulleys, biconvex lenses, ribbons, ravioli, flat pills, bicones, diamond disks, emarginate disks, elongated hexagonal disks, tacos, wrinkled prolate ellipsoids, wrinkled oblate ellipsoids, porous ellipsoid disks, . See, e.g., International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008.

In one embodiment, the device may be a syringe or vial in a kit with a transdermal insulin syringe, containing lyophilized or dried microparticles and a suspending agent such as sterile saline or phosphate buffered saline.

In one non-injectible embodiment, the device is applied to the skin or a mucosal surface (mouth, sublingual, rectal, vaginal). The device may have two components: (1) a display monitor/surface/signal release feature and (2) an analyte receiving/reaction chamber/surface. The two components may be contiguous or even a single dual purpose component.

Devices may be designed as rings, bracelets, watches, earrings, and other devices which are physically restrained at the site of contact, and/or incorporated into a bandage or wound dressing.

The devices may be applied by application of an adhesive or physical restraint.

Skin adhesives range in degree and length of duration, and can be obtained from 3M, Johnson & Johnson, and a variety of other medical supply companies. These may be cyanoacrylates for long term wound closure, or lightly adhesive of the type found on wound coverings such as BANDAID®s. A UV-inpenetrable transparent skin patch is described in U.S. Pat. No. 5,811,108 to Goeringer, which can be utilized in making a suitable transdermal device.

The mucosal devices may be in the form of a polymeric device that is mucoadhesive. These may be particles that are sprayed onto the tissue, particularly when the reaction is detected by a color change, or a device such as a disk.

Chemical enhancers have been found to increase transdermal drug transport via several different mechanisms, including increased solubility of the drug in the donor formulation, increased partitioning into the SC, fluidization of the lipid bilayers, and disruption of the intracellular proteins (Kost and Langer, In Topical Drug Bioavailability, Bioequivalence, and Penetration; Shah and Maibech, ed. (Plennum, NY 1993) pp. 91-103 (1993)). See also U.S. Pat. No. 5,445,611 to Eppstein, et al.

Chemical enhancers have been found to increase drug transport by different mechanisms. Chemicals which enhance permeability through lipids are known and commercially available. For example, ethanol has been found to increase the solubility of drugs up to 10,000-fold (Mitragotri, et al. In Encl. of Pharm. Tech.: Swarbrick and Boylan, eds. Marcel Dekker 1995) and yield a 140-fold flux increase of estradiol, while unsaturated fatty acids have been shown to increase the fluidity of lipid bilayers (Bronaugh and Maiback, editors (Marcel Dekker 1989) pp. 1-12).

Examples of fatty acids which disrupt lipid bilayer include linoleic acid, capric acid, lauric acid, and neodecanoic acid, which can be in a solvent such as ethanol or propylene glycol. Evaluation of published permeation data utilizing lipid bilayer disrupting agents agrees very well with the observation of a size dependence of permeation enhancement for lipophilic compounds. The permeation enhancement of three bilayer disrupting compounds, capric acid, lauric acid, and neodecanoic acid, in propylene glycol was reported by Aungst, et al. Pharm. Res. 7, 712-718 (1990).

A comprehensive list of lipid bilayer disrupting agents is described in European Patent Application 43,738 (1982). Exemplary compounds are represented by the formula:

R—

### X

wherein R is a straight-chain alkyl of about 7 to 16 carbon atoms, a non-terminal alkenyl of about 7 to 22 carbon atoms, or a branched-chain alkyl of from about 13 to 22 carbon atoms, and X is —OH, —COOCH₃, —COOC₂H₅, —OCOCH₃, —SOCH₃, —P(CH₃)₂O, COOC₂H₄OC₂H₄OH, —COOCH(CHOH)₄CH₂OH, —COOCH₂CHOHCH₃, COOCH₂CH(OR″)CH₂OR″, —(OCH₂CH₂),ₙOH, —COOR″, or —CONR″₂ where R′ is —H, —CH₃, —C₂H₅, —C₂H₇ or —C₂H₄OH; R″ is —H, or a non-terminal alkenyl of about 7 to 22 carbon atoms; and m is 2-6; provided that when R″ is an alkenyl and X is —OH or —COOH, at least one double bond is in the cis-configuration.

Suitable solvents include water; diols, such as propylene glycol and glycerol; mono-alcohols, such as ethanol, propanol, and higher alcohols; DMSO; dimethylformamide; N,N-dimethylacetamide; 2-pyrrolidone; N-(2-hydroxyethyl) pyrrolidone, N-methylpyrrolidone, 1-dodecylazacycloheptan-2-one and other n-substituted-alkyl-azacycloalkyl-2-ones and other n-substituted-alkyl-azacycloalkyl-2-ones (azones).

U.S. Pat. No. 4,537,776 to Cooper contains a summary of prior art and background information detailing the use of certain binary systems for permeant enhancement. European Patent Application 43,738, also describes the use of selected diols as solvents along with a broad category of cell-envelope disordering compounds for delivery of lipophilic pharmacologically-active compounds. A binary system for enhancing metaclopramide penetration is disclosed in UK Patent Application GB 2,153,223 A, consisting of a monovalent alcohol ester of a C8-32 aliphatic monocarboxylic acid (unsaturated and/or branched if C18-32) or a C6-24 aliphatic monoalcohol (unsaturated and/or branched if C14-24) and an N-cyclic compound such as 2-pyrrolidone or N-methylpyrrolidone.

Combinations of enhancers consisting of diethylene glycol monoethyl or monomethyl ether with propylene glycol monolaurate and methyl laurate are disclosed in U.S. Pat. No. 4,973,468 for enhancing the transdermal delivery of steroids such as progestogens and estrogens. A dual enhancer consisting of glycerol monolaurate and ethanol for the transdermal delivery of drugs is described in U.S. Pat. No. 4,820,720. U.S. Pat. No. 5,006,342 lists numerous enhancers for transdermal drug administration consisting of fatty acid esters or fatty alcohol ethers of C₂ to C₄ alkanediols, where each fatty acid/alcohol portion of the ester/ether is of about 8 to 22 carbon atoms. U.S. Pat. No. 4,863,970 discloses penetration-enhancing compositions for topical application including an active permeant contained in a penetration-enhancing vehicle containing specified amounts of one or more cell-envelope disordering compounds such as oleic acid, oleyl alcohol, and glycerol esters of oleic acid; a C₂ or C₃ alkanol and an inert diluent such as water.

Other chemical enhancers, not necessarily associated with binary systems, include dimethylsulfoxide (DMSO) or aqueous solutions of DMSO such as those described in U.S. Pat. No. 3,551,554 to Herschler; U.S. Pat. No. 3,711,602 to Herschler; and U.S. Pat. No. 3,711,606 to Herschler, and the azones (n-substituted-alkyl-azacycloalkyl-2-ones) such as noted in U.S. Pat. No. 4,557,943 to Cooper.

Some chemical enhancer systems may possess negative side effects such as toxicity and skin irritations. U.S. Pat. No. 4,855,298 discloses compositions for reducing skin irritation caused by chemical enhancer-containing compositions having skin irritation properties with an amount of glycerin sufficient to provide an anti-irritating effect.

Ultrasound with polyethylene glycol 200 dilaurate (PEG), isopropyl myristate (IM), and glycerol trioleate (GT) results in corticosterone flux enhancement values of only 2, 5, and 0.8, relative to the passive flux from PBS alone. However, 50% ethanol and LA/ethanol significantly increase corticosterone passive fluxes by factors of 46 and 900, indicating that the beneficial effects of chemical enhancers and therapeutic ultrasound can be effectively combined. Ultrasound combined with 50% ethanol produces a 2-fold increase in corticosterone transport above the passive case, but increase by 14-fold the transport from LA/Ethanol.

Ultrasound, mechanical abrasion and/or electrical fields can be used to enhance transdermal transfer. Echo Therapeutics, Franklin, Mass. has a SonoPrep® system that includes ultrasound-based skin permeation technology for a non-invasive and painless method of enhancing the flow of molecules across the skin's membrane for up to 24 hours. The SonoPrep system and its method of use are described in a variety of U.S. patents, including U.S. Pat. Nos. 6,190,315; 6,234,990; 6,491,657; 6,620,123.

Echo's application of ultrasonic energy creates reversible channels in the skin through which large molecules can be delivered or removed for analysis. This use of ultrasound technology makes it possible for painless and transdermal drug delivery or analyte extraction. The SonoPrep® system operates by transferring a low level of ultrasound energy for a short time from the hand piece, causing the outer most layer of skin (stratum corneum) to become permeable. The size of the sonication site is typically 0.8 cm². Echo has conducted studies to demonstrate that skin conductivity is significantly enhanced and that the enhancement lasts for several hours. The SonoPrep® system provides real-time skin conductance feedback. SonoPrep® measures the increase in skin conductance (or decrease in skin impedance) during the application of ultrasound and stops the sonocation procedure when the desired level of conductance has been achieved. This technology can be incorporated into the methods and compositions described herein to provide rapid easy one-step monitoring.

Monitors can be the particles that are embedded into the bandage, in an ointment or cream, or a reservoir type device having an area containing color changing chromophores, LEDs, liquid crystal display, or other material incorporated into the device itself. Liquid crystals (LC), as described above, can be bioerodible or non-bioerodible. Representative non-mesogenic, bioerodible polymers include polylactic acid, polylactide-co-glycolide, polycaprolactones, polyvaleric acid, polyorthoesters, polysaccharides, polypeptides, and certain polyesters. Representative mesogenic, bioerodible polymers include some polyanhydrides and polybutylene terephthalate. Examples of non-mesogenic, non-erodible polymers include polyethylene, polypropylene, polystyrene, and polytherephthalic acid. The polymer can be water-soluble or water-insoluble. These can be used in the controlled release or retention of substances encapsulated in the LC polymers. The polymer can be in a variety of forms including films, film laminants, and microparticles. In one embodiment, the LC polymers are used to encapsulate therapeutic, diagnostic, or prophylactic agents for use in medical or pharmaceutical applications. Other substances which can be encapsulated include scents such as perfumes, flavoring or coloring agents, sunscreen, and pesticides.

The LC polymer can be made in a variety of forms including films, film laminants, coatings, membranes, microparticles, slabs, extruded forms, and molded forms. The LC polymers can be combined with each other, with non-LC polymers, or with other materials such as metals, ceramics, glasses, or semiconductors, the latter typically in the form of coatings. The polymers can be fabricated into articles and then treated to induce the LC state, or the LC state can be induced and then articles formed from the LC polymer. Compositions that include the LC polymers can be monolithic or layered. The term “monolithic” is used herein to describe a continuous phase having imbedded structures, rather than layers. The LC polymers can be prepared separately and then mixed with other materials in a process that does not change the transition temperature. LC polymers can be used in display systems, such as for computers, and in message systems wherein a message can be displayed or hidden from view based on changes in the opacity/transparence of the LC polymer which occur with changes in the crystal structure of the material. LC polymers also can be used in product packaging. Another application for the LC polymers is in temperature sensing devices, for example. In one medical application, the sensor is attached to the skin to provide a temperature map indicating local temperature variations. Such devices are useful, for example, in the diagnosis of certain medical ailments, such as tumors, or areas of infection or inflammation or poor circulation which have a temperature different from the surrounding healthy tissue.

The device may be applied to a patient's oral cavity, for example, to the lingual and sub-lingual regions of the oral cavity. The underside and base of the tongue, as well as the base of the oral cavity beneath the tongue, are highly variegated and vascularized, containing capillaries close to the surface, which presents a considerable surface area to allow for transfer of analyte for detection and measurement.

The device may be in the form of a film, patch or other adhesive that adheres to the sublingual space, trapping the analyte in the device. Alternatively a powdered composition containing micro- or nano-particles may be delivered to the oral cavity, such as to the upper surface of the tongue, e.g., to the sublingual space.

Devices which adhere to mucosal surfaces and dissolve or otherwise disintegrate over time, delivering drug into the mouth of the patient in a sustained fashion, or release can be adapted for use as described herein. The device may contain at least one surface with a composition that exhibits good adherence to human oral mucosa. The device may be formed of a bioadhesive material or have one or more surfaces coated or formed of a bioadhesive material which adheres to a mucosal surface in the oral cavity, vaginal or rectal areas.

Buccal tablets are known. See, for example, in U.S. Pat. Nos. 4,740,365 and 4,764,378.

Adhesives for use with non-mucosal adhesive devices that adhere to mucosal surfaces are known to the art. Polyacrylic acids and polyisobutylenes have been disclosed as components of such adhesives. For example, U.S. Pat. No. 3,339,546 to Chen discloses a bandage that is said to adhere to moist surfaces of the oral cavity and comprises a medicament and a hydrocolloid (carboxypolymethylene (i.e., polyacrylic acid)) incorporated in a natural or synthetic gum-like substance. U.S. Pat. No. 4,615,697 to Robinson discloses a composition including a bioadhesive and a treating agent. The bioadhesive is a water-swellable but water insoluble, fibrous, crosslinked, carboxy-functional polymer containing a plurality of repeating units of which at least about 80% contain at least 1 carboxy functionality, and about 0.05 to about 1.5% of a cross-linking agent substantially free from polyalkenyl polyether. U.S. Pat. No. 4,253,460 to Chen et al. discloses an adhesive composition consisting of a mixture of a hydrocolloid gum, a pressure sensitive adhesive, and a cohesive strengthening agent. The pressure sensitive adhesive component can be a mixture of three to five parts of a polyisobutylene with a viscosity average molecular weight of about 36,000 to about 53,000 and one part of an elastomer such as a polyisobutylene with a viscosity average molecular weight of about 1,150,000 to about 1,600,000. U.S. Pat. No. 4,740,365 to Yukimatsu et al. discloses a sustained-release preparation comprising an active ingredient and a mixture of two polymer components, the first of which comprises polyacrylic acid or a pharmaceutically acceptable salt thereof, and the second is polyvinylpyrrolidone, polyvinyl alcohol, polyethylene glycol, alginic acid, or a pharmaceutically acceptable salt of alginic acid. CARBOPOL® resins are among the polymers said to be suitable members of the first-mentioned class of polymers. U.S. Pat. No. 4,772,470 to Inoue, et al. discloses an oral bandage comprising a mixture of a polyacrylic acid and a vinyl acetate polymer in a compatible state. This bandage is said to exhibit strong adhesion of long duration when applied to oral mucosa or teeth.

Mucoadhesive polymers are defined as polymers that have an adherence to living mucosal tissue of at least about 110 N/m² of contact area (11 mN/cm²). A suitable measurement method is set forth in U.S. Pat. No. 6,235,313 to Mathiowitz et al. Polyanhydrides are one type of mucoadheisve polymer. The mechanism causing the anhydride polymers or oligomers to be bioadhesive is believed to be due to a combination of the polymer's hydrophobic backbone, coupled with the presence of carboxyl groups at the ends. Interaction of charged carboxylate groups with tissue has been demonstrated with other bioadhesives. In particular, pharmaceutical industry materials considered to be bioadhesive typically are hydrophilic polymers containing carboxylic acid groups, and often hydroxyl groups as well. The industry standard is often considered to be CARBOPOL™ (a high molecular weight poly(acrylic acid)). Other classes of bioadhesive polymers are characterized by having moderate to high densities of carboxyl substitution. The relatively hydrophobic anhydride polymers frequently demonstrate superior bioadhesive properties when compared with the hydrophilic carboxylate polymers.

Suitable polyanhydrides include polyadipic anhydride, poly fumaric anhydride, polysebacic anhydride, polymaleic anhydride, poly malic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different molar ratios.

Natural adhesives for underwater attachment of mussels, other bivalves and algae to rocks and other substrates are known (see U.S. Pat. No. 5,574,134 to Waite, U.S. Pat. No. 5,015,677 to Benedict et al., and U.S. Pat. No. 5,520,727 to Vreeland et al.). These adhesives are polymers containing poly(hydroxy-substituted) aromatic groups. In mussels and other bivalves, such polymers include dihydroxy-substituted aromatic groups, such as proteins containing 3,4-dihydroxyphenylalanine (DOPA). In algae, diverse polyhydroxy aromatics such as phloroglucinol and tannins are used. In adhering to an underwater surface, the bivalves secrete a preformed protein that adheres to the substrate thereby linking the bivalve to the substrate. After an initial adherence step, the natural polymers are typically permanently crosslinked by oxidation of adjacent hydroxyl groups. The attachment of DOPA to different polymeric backbones is described in U.S. Pat. No. 4,908,404 to Benedict et al. and U.S. Publication No. 2005/0201974 to Schestopol et al. Suitable mucoadhesive polymers include Other mucoadhesive polymers include DOPA-maleic anhydride co polymer; isopthalic anhydride polymer; DOPA-methacrylate polymers; and DOPA-cellulosic based polymers.

Bioadhesive materials contain a polymer with a catechol functionality. The molecular weight of the bioadhesive materials and percent substitution of the polymer with the aromatic compound may vary greatly. The degree of substitution varies based on the desired adhesive strength, it may be as low as 10%, 20%, 25%, 50%, or up to 100% substitution. On average at least 50% of the monomers in the polymeric backbone are substituted with at least one aromatic group. In some cases, 75-95% of the monomers in the backbone are substituted with at least one aromatic group or a side chain containing an aromatic group. In one embodiment, on average 100% of the monomers in the polymeric backbone are substituted with at least one aromatic group or a side chain containing an aromatic group. The resulting bioadhesive material is a polymer with a molecular weight ranging from about 1 to 2,000 kDa. The polymer that forms that backbone of the bioadhesive material may be any non-biodegradable or biodegradable polymer. In some cases, the polymer is a hydrophobic polymer. In one embodiment, the polymer is a biodegradable polymer and is used to form an oral dosage formulation.

Examples of biodegradable polymers include synthetic polymers such as poly hydroxy acids, such as polymers of lactic acid and glycolic acid, polyanhydrides, poly(ortho)esters, polyesters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In some instances, these materials degrade either by enzymatic hydrolysis or exposure to water in vivo, by surface or bulk erosion. The foregoing materials may be used alone, as physical mixtures (blends), or as co-polymers.

Mucoadhesive materials also include poly(fumaric acid:sebacic acid), as described in U.S. Pat. No. 5,955,096 to Mathiowitz et al., incorporating oligomers and metal oxides polymer to enhance the ability of the polymer to adhere to a tissue surface such as a mucosal membrane, as described in U.S. Pat. No. 5,985,312 to Jacob et al. In one embodiment, the polymer is a biodegradable polymer.

Microparticles and nanoparticles can be prepared using a variety of techniques known in the art. The functional groups used to bind or complex the analyte can be introduced prior to microparticle formation (e.g., monomers can be functionalized with one or more functional groups for binding or complexing the analyte) or the functional groups can be introduced after microparticle formation (e.g., by functionalizing the surface of the microparticle with reactive functional groups). The microparticles may optionally have encapsulated therein one or more core materials. In one embodiment, the microparticles or nanoparticles should be present in an effective amount to provide a signal detectable to the user without the need for additional equipment. For example, the microparticles and/or nanoparticles should be present in an effective amount to provide a change in taste, smell, shape, and/or color upon binding or complexing the analyte that is easily detectable by the user.

The following are representative methods for forming microparticles and nanoparticles. Techniques other than those described below may also be used to prepare microparticles and/or nanoparticles.

Techniques for forming anisotrophic particles or fibers can be found in U.S. patent application Ser. No. 11/272,194, filed Nov. 10, 2005, entitled “Multi-Phasic Nanoparticles,” by Lahann, et al., published as U.S. Patent Application Publication No. 2006/0201390 on Sep. 14, 2006; or priority to U.S. patent application Ser. No. 11/763,842, filed Jun. 15, 2007, entitled “Multiphasic Biofunctional Nano-Components and Methods for Use Thereof,” by Lahann, published as U.S. Patent Application Publication No. 2007/0237800 on Oct. 11, 2007.

In one embodiment, the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent evaporated, leaving solid nanoparticles. The resulting nanoparticles are washed with water and dried overnight in a lyophilizer. Nanoparticles with different sizes (0.5-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

In one embodiment using solvent removal, the polymer is dissolved in a volatile organic solvent like methylene chloride. The mixture is suspended by stirring in an organic oil (such as silicon oil) to form an emulsion. Unlike solvent evaporation, this method can be used to make nanoparticles from polymers with high melting points and different molecular weights. Nanoparticles that range between 1-300 microns can be obtained by this procedure. The external morphology of spheres produced with this technique is highly dependent on the type of polymer used.

Another embodiment uses spray-drying, where the polymer is dissolved in organic solvent. The solution or the dispersion is then spray-dried. Typical process parameters for a mini-spray drier (Buchi) are as follows: polymer concentration=0.04 g/mL, inlet temperature=−24° C., outlet temperature=13-15° C., aspirator setting=15, pump setting=10 mL/minute, spray flow=600 Nl/hr, and nozzle diameter=0.5 mm Nanoparticles ranging between 1-10 microns can be obtained with a morphology which depends on the type of polymer used.

For interfacial polycondensation, one monomer is dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

Microspheres can be formed from polymers using a phase inversion method wherein a polymer is dissolved in a “good” solvent and the mixture is poured into a strong non solvent for the polymer, to spontaneously produce, under favorable conditions, polymeric microspheres. The method can be used to produce nanoparticles in a wide range of sizes, including, for example, about 100 nanometers to about 10 microns. Exemplary polymers which can be used include polyvinylphenol and polylactic acid. In the process, the polymer is dissolved in an organic solvent and then contacted with a non solvent, which causes phase inversion of the dissolved polymer to form small spherical particles, with a narrow size distribution optionally incorporating an antigen or other substance.

In phase separation, the polymer is dissolved in a solvent to form a polymer solution. While continually stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, forming a particles with a polymeric shell.

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

Nanoparticles made of gel-type polymers, such as alginate and hyaluronic acid, can be produced through traditional ionic gelation techniques. The polymers are first dissolved in an aqueous solution and then extruded through a microdroplet forming device, which in some instances employs a flow of nitrogen gas to break off the droplet. A slowly stirred (approximately 100-170 RPM) ionic hardening bath is positioned below the extruding device to catch the forming microdroplets. The nanoparticles are left to incubate in the bath for twenty to thirty minutes in order to allow sufficient time for gelation to occur. Nanoparticle size is controlled by using various size extruders or varying either the nitrogen gas or polymer solution flow rates. Chitosan nanoparticles can be prepared by dissolving the polymer in acidic solution and crosslinking it with tripolyphosphate. Carboxymethyl cellulose (CMC) nanoparticles can be prepared by dissolving the polymer in acid solution and precipitating the nanoparticle with lead ions. In the case of negatively charged polymers (e.g., alginate, CMC), positively charged ligands (e.g., polylysine, polyethyleneimine) of different molecular weights can be ionically attached.

Other methods known in the art that can be used to prepare nanoparticles include, but are not limited to, polyelectrolyte condensation (see Suk et al., Biomaterials, 27, 5143-5150 (2006)); single and double emulsion (probe sonication); nanoparticle molding, and electrostatic self-assembly (e.g., polyethylene imine-DNA or liposomes).

Electrospraying or electrospinning techniques can be used to prepare particles. In some cases, two or more fluid streams (including liquid jets) are combined together such that the two or more fluid streams contact over spatial dimensions sufficient to form a composite stream. In some cases, there is little or no mixing of the two or more fluid streams within the composite stream. In some variations, the fluid streams are electrically conductive, and in certain cases, a cone-jet may be formed by combining the two or more fluid streams under the influence of an electric field.

In some cases, the composite stream is directed at a substrate, e.g., by the application of a force field such as an electric field. For instance, if the composite stream is charged, an electric field may be used to urge the composite stream towards a substrate. The composite stream may be continuous or discontinuous in some cases, e.g., forming a series of droplets (which may be spherical or non-spherical). In some cases, the composite stream is hardened prior to and/or upon contact with the substrate. For example, the composite stream may be urged towards the substrate under conditions in which at least a portion of the composite stream (e.g., a solvent) is able to evaporate, causing the remaining stream to harden, e.g., to form particles, spheres, rods, fibers, . In some variations, the composite stream fragments in droplets that can lead to particle, sphere, rod, and/or fiber formation.

Blood glucose, insulin, hormone levels are all representative normal analytes to measure, where critical levels trigger a signal. The reaction entities may be used to determine pH or metal ions, proteins, nucleic acids (e.g. DNA, RNA, etc.), drugs, sugars (e.g., glucose), hormones (e.g., estradiol, estrone, progesterone, progestin, testosterone, androstenedione, etc.), carbohydrates, or other analytes of interest. Examples of analytes to be measured include glucose (e.g., for diabetics); sodium, potassium, chloride, calcium, magnesium, and/or bicarbonate (e.g., to determine dehydration); gases such as carbon dioxide or oxygen; pH; metabolites such as urea, blood urea nitrogen or creatinine; hormones such as estradiol, estrone, progesterone, progestin, testosterone, androstenedione, etc. (e.g., to determine pregnancy, illicit drug use, etc.); or cholesterol.

Other variables can include pH changes, which may indicate disease, yeast infection, periodontal disease at a mucosal surface, oxygen or carbon monoxide levels which indicate lung dysfunction, and drug levels, both legal prescription levels of drugs such as coumadin and illegal such as cocaine or nicotine.

In one embodiment, these analytes are measured as an “on/off” or “normal/abnormal” situation, where the device indicates a change. This might be that insulin is needed; a trip to the doctor to check cholesterol; ovulation is occurring; kidney dialysis is needed; drug levels are present (especially in the case of illegal drugs) or too high/too low (important in care of geriatrics in particular in nursing homes).

Examples of abnormal analytes include those indicative of disease, such as cancer specific markers such as CEA and PSA, viral and bacterial antigens, and autoimmune indicators such antibodies to double stranded DNA, indicative of Lupus.

Various pathogens such as bacteria or viruses, and/or markers produced by such pathogens may be detected, for example, by reaction with an antibody directed at a marker produced by bacteria.

In the majority of these cases, the indicator is set as a “warning light,” where the individual is then referred to a physician for further followup.

For example, anisotropic particles are prepared comprising a biocompatible polymer such as polyethylene oxide (PEO), or a polymer of polylactic acid (PLA) and/or polyglycolic acid (PGA). The first half of the particles contains a reactive partner to a pathogen, such as an antibody to the pathogen and/or a marker produced by the pathogen (e.g., a protein). As a specific example, the pathogen may be anthrax and the antibody may be an antibody to anthrax spores. As another example, the pathogen may be a Plasmodia (some species of which causes malaria) and the antibody may be an antibody that recognizes the Plasmodia. In some cases, these may be soluble molecules that can enter the interstitial fluid or the blood. The first half also contains a first colorant, which may be green, e.g., such as fluorescein or GFP. The second half may contain a second colorant, which may be red, e.g., rhodamine.

The particles are suspended in saline and injected into the skin of a human subject. The particles may be injected into the dermis and/or the epidermis, e.g., to form a “mark” within the skin In the absence of the pathogen, no aggregation of the particles occurs, and the particles are present in a random orientation within the skin; thus, one sees a mixture of red and green (e.g., giving a brown-colored appearance). In the presence of the pathogen (or pathogen marker), however, some aggregation of the particles occurs, such that the particles orient around the pathogen, where the first half of the particles orients to the pathogen due to the presence of the pathogen reactive partner. Thus, visually, the second colorant will dominant when the particles are aggregated; thus, one sees a brighter red colored appearance.

Other variables may include exposure to elevated carbon monoxide, which could be from an external source or due to sleep apnea, too much heat (important in the case of babies whose internal temperature controls are not fully self-regulating) or from fever.

The devices are applied and then the result detected based on the site of administration and the device. In general, the devices, whether a suspension of microparticles or a device containing a surface with a detectable signal as well as a reaction chamber, will be administered topically to the skin, injected into the dermis or subcutaneously, or administered to a mucosal surface.

The device may be applied as a bandage, a plastic “watch,” “bracelet,” or “ring,” or a specifically designed apparatus for direct application to the skin The device may be secured physically by restraints or by an adhesive. The reactive agents may also be contained within a cream or a lotion which can be rubbed onto the skin to deliver the particles. The cream or lotion may contain, for instance, an emulsion of a hydrophobic and a hydrophilic material (e.g., oil and water), distributed in any order (e.g., oil-in-water or water-in-oil), and the particles may be present in any one or more of the emulsion phases. In some cases, the particles may be administered by a medical practitioner; in other cases, however, the particles may be self-administered.

In some or all cases, the skin may first be treated with a transdermal penetration enhancer, mechanical abrasion or pressure or ultrasound.

The reactive agents may be delivered to any location within the skin (or below the skin), e.g., to the epidermis, to the dermis, subcutaneously, or intramuscularly, but to the epidermis or subcutaneously to facilitate easily discernible detection. In some cases, a “depot” of reactive agents may be formed within the skin, and the depot may be temporary or permanent. The reactive agents within the depot may eventually degrade or disperse (e.g., if the reactive agents or carriers bound thereto are biodegradable or cleaved at time of reaction), enter the bloodstream, or be sloughed off to the environment.

In some cases, especially if the reactive agents are colored, the reactive agents after delivery may give the appearance of a “tattoo” or a permanent mark within the skin, and the tattoo or other mark may be of any color and/or size. In one embodiment, anisotropic particles such as those described above that are able to bind glucose may be delivered into the skin of a subject, and such particles, after deposition within the skin, may react to the presence or absence of glucose by exhibiting a change in color. The particles may exhibit a color change based on the presence or absence of glucose, and/or the concentration of glucose. For instance, the particles may exhibit a first color (e.g., green) when not aggregated, and a second color (e.g., red or brown) when aggregated, or the particles may be invisible when not aggregated, but visible (e.g., exhibiting a color) when aggregated. The particles may be, for example, anisotropic particles having a first surface region having a first color (e.g., green) and a second surface region having a second color (e.g., red), and the first surface region may contain a reactive partner to glucose. At low levels of glucose, the particles may exhibit a combination of the first and second colors, while at higher levels of glucose, the particles may exhibit more of the second color.

As one non-limiting example, in one set of embodiments, a hypodermic needle or similar device may be used to deliver particles into various tissues. Hypodermic needles are well-known to those of ordinary skill in the art, and can be obtained with a range of needle gauges. Examples of needles include the 20-30 gauge range, or the needle may be 32 gauge, 33 gauge, 34 gauge, etc.

In another set of embodiments, microneedles such as those disclosed in U.S. Pat. No. 6,334,856, may be used to deliver the particles to the dermis and/or the epidermis, depending on the shape and/or size of the microneedles, as well as the location of delivery. The microneedles may be formed from any suitable material, e.g., metals, ceramics, semiconductors, organics, polymers, and/or composites. Examples include, but are not limited to, pharmaceutical grade stainless steel, gold, titanium, nickel, iron, gold, tin, chromium, copper, alloys of these or other metals, silicon, silicon dioxide, and polymers, including polymers of hydroxy acids such as lactic acid and glycolic acid polylactide, polyglycolide, polylactide-co-glycolide, and copolymers with polyethylene glycol, polyanhydrides, polyorthoesters, polyurethanes, polybutyric acid, polyvaleric acid, polylactide-co-caprolactone, polycarbonate, polymethacrylic acid, polyethylenevinyl acetate, polytetrafluorethylene, or polyesters. In some cases, the particles may be delivered via the microneedles; in other cases, however, the microneedles may be first applied to the skin and removed to create passages through the skin (e.g., through the stratum corneum, which is the outermost layer of the skin), then the particles subsequently applied to the skin.

One or more distinct and continuous pathways can be created through the interior of microneedles. In one example, the microneedle has a single annular pathway along the center axis of the microneedle. This pathway can be achieved by initially chemically or physically etching the holes in the material and then etching away microneedles around the hole. Alternatively, the microneedles and their holes can be made simultaneously or holes can be etched into existing microneedles. As another option, a microneedle form or mold can be made, then coated, and then etched away, leaving only the outer coating to form a hollow microneedle. Coatings can be formed either by deposition of a film or by oxidation of the silicon microneedles to a specific thickness, followed by removal of the interior silicon. Also, holes from the backside of the wafer to the underside of the hollow needles can be created using a front-to-backside infrared alignment followed by etching from the backside of the wafer.

One method for hollow needle fabrication is to replace the solid mask used in the formation of solid needles by a mask that includes a solid shape with one or more interior regions of the solid shape removed. One example is a “donut-shaped” mask.

Using this type of mask, interior regions of the needle are etched simultaneously with their side walls. Due to lateral etching of the inner side walls of the needle, this may not produce sufficiently sharp walls. In that case, two plasma etches may be used, one to form the outer walls of the microneedle (i.e., a standard etch), and one to form the inner hollow core (which is an extremely anisotropic etch, such as in inductively-coupled-plasma “ICP” etch). For example, the ICP etch can be used to form the interior region of the needle followed by a second photolithography step and a standard etch to form the outer walls of the microneedle.

Alternatively, this structure can be achieved by substituting the chromium mask used for the solid microneedles by a silicon nitride layer on the silicon substrate covered with chromium. Solid microneedles are then etched, the chromium is stripped, and the silicon is oxidized to form a thin layer of silicon dioxide on all exposed silicon surfaces. The silicon nitride layer prevents oxidation at the needle tip. The silicon nitride is then stripped, leaving exposed silicon at the tip of the needle and oxide-covered silicon everywhere else. The needle is then exposed to an ICP plasma which selectively etches the inner sidewalls of the silicon in a highly anisotropic manner to form the interior hole of the needle.

Another example uses the solid silicon needles described previously as “forms” or molds around which the actual needle structures are deposited. After deposition, the forms are etched away, yielding the hollow structures. Silica needles or metal needles can be formed using different methods. Silica needles can be formed by creating needle structures similar to the ICP needles described above prior to the oxidation described above. The wafers are then oxidized to a controlled thickness, forming a layer on the shaft of the needle form which will eventually become the hollow microneedle. The silicon nitride is then stripped and the silicon core selectively etched away (e.g., in a wet alkaline solution) to form a hollow silica microneedle.

In another example, an array of hollow silicon microtubes is made using deep reactive ion etching combined with a modified black silicon process in a conventional reactive ion etcher. First, arrays of circular holes are patterned through photoresist into SiO₂, such as on a silicon wafer. Then the silicon can be etched using deep reactive ion etching (DRIE) in an inductively coupled plasma (ICP) reactor to etch deep vertical holes. The photoresist was then removed. Next, a second photolithography step patterns the remaining SiO₂ layer into circles concentric to the holes, leaving ring shaped oxide masks surrounding the holes. The photoresist is then removed and the silicon wafer again deep silicon etched, such that the holes are etched completely through the wafer (inside the SiO₂ ring) and simultaneously the silicon is etched around the SiO₂ ring leaving a cylinder.

This latter example can also be varied to produce hollow, tapered microneedles. After an array of holes is fabricated as described above, the photoresist and SiO₂ layers are replaced with conformal DC sputtered chromium rings. The second ICP etch is replaced with a SF₆/O₂ plasma etch in a reactive ion etcher (RIE), which results in positively sloping outer sidewalls.

Metal needles can be formed by physical vapor deposition of appropriate metal layers on solid needle forms, which can be made of silicon using the techniques described above, or which can be formed using other standard mold techniques such as embossing or injection molding. The metals are selectively removed from the tips of the needles using electropolishing techniques, in which an applied anodic potential in an electrolytic solution will cause dissolution of metals more rapidly at sharp points, due to concentration of electric field lines at the sharp points. Once the underlying silicon needle forms have been exposed at the tips, the silicon is selectively etched away to form hollow metallic needle structures. This process could also be used to make hollow needles made from other materials by depositing a material other than metal on the needle forms and following the procedure described above.

nanoBioSciences of Alameda, Calif. has developed a proprietary drug delivery patch system, dubbed AdminPatch, based on tiny microneedles form pressed out of standard metallic film. The AdminPatch system is an advanced microneedle transdermal delivery technology that painlessly and instantaneously forms hundreds of tiny aqueous channels (‘micropores’) through the stratum corneum and epidermis, the outer resistive surface layers of skin. Proteins and water-soluble molecules can enter the body through these aqueous micropores for either local effect, or by entering the circulation, for systemic effect. The created aqueous channels stay constantly open while AdminPatch is applied on the skin and, therefore, enable the rapid, sustained, and efficient delivery of drugs through these aqueous channels formed in the skin surface.

The AdminPatch system is comprised of a single-use disposable AdminPatch and a re-useable handheld Applicator. The disposable AdminPatch contains the proprietary microneedle array laminated on a conventional transdermal drug-in-adhesive patch.

Another disposable adhesive microneedle patch is available from Theraject, Inc., Menlo Park, Calif.

Hollow, porous, or solid microneedles can be provided with longitudinal grooves or other modifications to the exterior surface of the microneedles. Grooves, for example, should be useful in directing the flow of molecules along the outside of microneedles. Polymeric microneedles are also made using microfabricated molds. For example, the epoxy molds can be made as described above and injection molding techniques can be applied to form the microneedles in the molds. In some cases, the polymer is a biodegradable polymer such as those described above.

The depth of penetration of particles into the skin is determined, at least in part, by the length of the microneedles. For instance, longer microneedles may be used to penetrate the skin to the level of the dermis, such that at least some of the particles are delivered to the dermis, while shorter microneedles may only penetrate the skin to the level of the epidermis, such that most (if not all) of the particles are delivered into the epidermis.

Pressurized fluids may be used to deliver particles, for instance, using a jet injector or a “hypospray.” Typically, such devices produce a high-pressure “jet” of liquid or powder (e.g., a biocompatible liquid, such as saline) that drives the particles into the skin, and the depth of penetration may be controlled, for instance, by controlling the pressure of the jet. The pressure may come from any suitable source, e.g., a standard gas cylinder or a gas cartridge. See, e.g., U.S. Pat. No. 4,103,684. Pressurization of the liquid may be achieved using compressed air or gas, for instance, by a pressure hose from a large cylinder, or from a built-in gas cartridge or small cylinder.

The depth of penetration of the skin may be controlled by controlling the degree of pressurization of the liquid. In general, higher pressures allow deeper penetration through the skin. Thus, at relatively low pressures, the particles are able to penetrate into the epidermis; at relatively higher pressures, at least some of the particles will penetrate into the dermis of the skin as well.

The devices may be applied to a mucosal surface by spraying a powder, or application of a mucoadhesive device to the tissue. This may be sublingual, buccal, vaginal, rectal, or even intra-nasal.

The signal can be detected either on the surface or within the device, or in the vicinity of the device. The situation with anisotrophic particles is discussed above.

These are used to generate a pattern or color which is indicative of the presence and/or amount of analyte. The density, shape, color, or intensity of the pattern or color may provide a yes-no type answer or may be graduated to provide quantitative amounts. This could also be effected by exposure to a pH or temperature change in some embodiments. Other patterns include, for example, + and − signs, arrows (e.g., up arrows or down arrows), faces (smiley, neutral, sad), etc., or the like.

The device or skin or tissue surface may change in feel when there is a reaction. For example, shape memory polymers may say “OK” when the cholesterol level is below 150 mg/dl. These may change to ready “HIGH” when the cholesterol level exceeds 200 mg/dl. The device may be blank or lack definition at values between these levels.

The device may change taste or smell when reacted with analyte. This may result in a smell such as a food odor being release as a function of a pH or temperature change which released encapsulated scent, or, in the case of a mucosal device, which releases food flavoring such as mint or cinnamon. In one set of embodiments, FDA GRAS ingredients may be used as signals.

The devices provide a method of determining the presence or amount of analyte by administering to the site where analyte is to be measured a single step diagnostic device for determination of the presence and/or amount of an analyte in a person, wherein the device is administered topically, under or within the skin or mucosal surface, and the device includes: reactive agents which react with an analyte to be detected at the site of administration and agents which generate a signal that can be detected visually, by feel, by smell, or by taste, at the site of reaction with the analyte, without reference to an external or secondary device or reference sample.

These may be applied to the skin or mucosa to measure a change in temperature indicative of disease or inflammation. In some embodiments, the device may be colorless or a color indicative of normal temperature (for example, green), or the device will display a message such as “OK.” In the event the temperature exceeds a certain level, such as 101° F., the color changes (for example, yellow for caution or red for warning or critical) or the message changes (for example, if shape memory polymers are used) to read “HOT.” These are particularly useful in a setting such as a day care, where there are a number of babies or young children to supervise, and fevers can occur rapidly.

In another embodiment, the devices may be used to measure a decrease in blood oxygen, or measure the amount of molecules such as glucose, cholesterol, triglycerides, cancer markers, or infectious agents, by providing reactive agents that specifically react with the molecules, and signal generating agents which produce signal in an amount correlated with the amounts of the molecules that react. Alternatively, analogous to the temperature monitor, a pre-set level can be used to create a message that says “C high,” for example, or “insulin!”, for example, which effects a color change.

As discussed above, the devices may, instead of a color change or message change, change shape, emit a scent or flavor, or otherwise notify the person of a need to seek further information. In some cases, this might be to seek medical attention where the indicator of a disorder can be confirmed and appropriate medical intervention obtained. In the case of temperature indicative of a fever, the caregiver might measure the temperature using a standard thermometer. In the case of a hormone change, indicative of pregnancy or ovulation, an ELISA test might be performed using a urine sample. In the case of high glucose, this could be confirmed using a standard glucose monitor and a blood sample.

Such devices are not meant as a final diagnostic, but as an indicator of a condition that requires further follow up.

Non-biological applications are also contemplated in other embodiments of the invention. For example, the particles or other agents that exhibits a determinable change when exposed to different concentrations or amounts of oxygen may be administered to a liquid or a solid sample to determine the oxygen concentration that the sample is exposed to. For example, the particles may be contained within an article that is placed with a food sample, a drug, or a pharmaceutical preparation, a consumer item, or other sample where exposure to oxygen is important. The article may exhibit a first color (or other determinable property, e.g., temperature, odor, etc., as discussed herein) at a first (e.g., acceptable) oxygen concentration, but a second color at a second (e.g., unacceptable oxygen concentration). In such a manner, the condition of the sample may be readily assessed, e.g., by the human eye without the use of any equipment. As still another example, particles or other agents that exhibits a determinable change when exposed to different concentrations or amounts of oxygen may be administered to a reactor, e.g., one in which particularly reactive chemicals are being used. Thus, if the particles exhibits a certain color, temperature, odor, etc., then this would indicate that the reactor has reached a certain condition (e.g., a certain oxygen concentration), which may be a desired or undesired outcome, depending on the application. For example, certain chemical reactions may be desirably performed in the absence of oxygen; if the particles exhibit a certain color, indicating that there is a certain concentration of oxygen present, then the chemical reaction may have been compromised in some fashion.

As mentioned, certain aspects of the present invention are generally directed to particles such as anisotropic particles or colloids, which can be used in a wide variety of applications. The particles may include microparticles and/or nanoparticles. As discussed above, a “microparticle” is a particle having an average diameter on the order of micrometers (i.e., between about 1 micrometer and about 1 mm), while a “nanoparticle” is a particle having an average diameter on the order of nanometers (i.e., between about 1 nm and about 1 micrometer. The particles may be spherical or non-spherical, in some cases. For example, the particles may be oblong or elongated, or have other shapes such as those disclosed in U.S. patent application Ser. No. 11/851,974, filed Sep. 7, 2007, entitled “Engineering Shape of Polymeric Micro- and Nanoparticles,” by S. Mitragotri, et al.; International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008; U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007, each of which is incorporated herein by reference.

An “anisotropic” particle, as used herein, is one that is not spherically symmetric (although the particle may still exhibit various symmetries), although the particle may have sufficient asymmetry to carry out at least some of the goals of the invention as described herein. On the basis of the present disclosure, this will be clearly understood by those of ordinary skill in the art. The asymmetry can be asymmetry of shape, of composition, or both. As an example, a particle having the shape of an egg or an American football is not perfectly spherical, and thus exhibits anisotropy. As another example, a sphere painted such that exactly one half is red and one half is blue (or otherwise presents different surface characteristics on different sides) is also anisotropic, as it is not perfectly spherically symmetric, although it would still exhibit at least one axis of symmetry.

Accordingly, a particle may be anisotropic due to its shape and/or due to two or more regions that are present on the surface of and/or within the particle. For instance, the particle may include a first surface region and a second surface region that is distinct from the first region in some way, e.g., due to coloration, surface coating, the presence of one or more reaction entities, etc. The particle may include different regions only on its surface or the particle may internally include two or more different regions, portions of which extend to the surface of the particle. The regions may have the same or different shapes, and be distributed in any pattern on the surface of the particle. For instance, the regions may divide the particle into two hemispheres, such that each hemisphere has the same shape and/or the same surface area, or the regions may be distributed in more complex arrangements.

Non-limiting examples of anisotropic particles can be seen in U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007; or U.S. Provisional Patent Application Ser. No. 61/058,796, filed Jun. 4, 2008, entitled “Compositions and Methods for Diagnostics, Therapies, and Other Applications,” by D. L. Levinson, each of which is incorporated herein by reference.

The particles (which may be anisotropic, or not anisotropic) may be formed of any suitable material, depending on the application. For example, the particles may comprise a glass, and/or a polymer such as polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, the particles may comprise a ceramic such as tricalcium phosphate, hydroxyapatite, fluorapatite, aluminum oxide, or zirconium oxide. In some cases (for example, in certain biological applications), the particles may be formed from biocompatible and/or biodegradable polymers such as polylactic and/or polyglycolic acids, polyanhydride, polycaprolactone, polyethylene oxide, polybutylene terephthalate, starch, cellulose, chitosan, and/or combinations of these. In one set of embodiments, the particles may comprise a hydrogel, such as agarose, collagen, or fibrin. The particles may include a magnetically susceptible material in some cases, e.g., a material displaying paramagnetism or ferromagnetism. For instance, the particles may include iron, iron oxide, magnetite, hematite, or some other compound containing iron, or the like. In another embodiment, the particles can include a conductive material (e.g., a metal such as titanium, copper, platinum, silver, gold, tantalum, palladium, rhodium, etc.), or a semiconductive material (e.g., silicon, germanium, CdSe, CdS, etc.). Other particles potentially useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, or GaAs. The particles may include other species as well, such as cells, biochemical species such as nucleic acids (e.g., RNA, DNA, PNA, etc.), proteins, peptides, enzymes, nanoparticles, quantum dots, fragrances, indicators, dyes, fluorescent species, chemicals, small molecules (e.g., having a molecular weight of less than about 1 kDa), or the like.

As an example, certain particles or colloids such as gold nanoparticles can be coated with various agents, e.g., capable of interacting with oxygen. Such particles may associate with each other, or conversely, dissociate in such a manner that a change is conferred upon the light absorption property of the material containing the particles. This approach can also be used as a skin-based visual sensor, in one embodiment. A non-limiting example of a technique for identifying aggregates is disclosed in U.S. patent application Ser. No. 09/344,667, filed Jun. 25, 1999, entitled “Nanoparticles Having Oligonucleotides Attached Thereto and Uses Therefor,” by Mirkin, et al., now U.S. Pat. No. 6,361,944, issued Mar. 26, 2002.

The particles may also have any shape or size. For instance, the particles may have an average diameter of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm As discussed, the particles may be spherical or non-spherical. The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous. Other shapes are also possible, for instance, core/shell structures (e.g., having different compositions), rectangular disks, high aspect ratio rectangular disks, high aspect ratio rods, worms, oblate ellipses, prolate ellipses, elliptical disks, UFOs, circular disks, barrels, bullets, pills, pulleys, biconvex lenses, ribbons, ravioli, flat pills, bicones, diamond disks, emarginate disks, elongated hexagonal disks, tacos, wrinkled prolate ellipsoids, wrinkled oblate ellipsoids, porous ellipsoid disks, and the like. See, e.g., International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008, incorporated herein by reference.

In another aspect, the present invention is directed to a kit including one or more of the compositions previously discussed, e.g., a kit including a particle, a kit including a device for the delivery and/or withdrawal of fluid from the skin, a kit including a device able to create a pooled region of fluid within the skin of a subject, a kit including a device able to determine a fluid, or the like. A “kit,” as used herein, typically defines a package or an assembly including one or more of the compositions or devices of the invention, and/or other compositions or devices associated with the invention, for example, as previously described. For example, in one set of embodiments, the kit may include a device and one or more compositions for use with the device. Each of the compositions of the kit, if present, may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

U.S. Provisional Patent Application Ser. No. 61/058,796, filed Jun. 4, 2008, entitled “Compositions and Methods for Diagnostics, Therapies, and Other Applications,” by D. Levinson, is incorporated herein by reference. Also incorporated herein by reference are U.S. Provisional Patent Application Ser. No. 61/163,733, filed on Mar. 26, 2009, entitled “Determination of Tracers within Subjects,” by D. Levinson; U.S. Provisional Patent Application Ser. No. 61/163,750, filed on Mar. 26, 2009, entitled “Monitoring of Implants and Other Devices,” by D. Levinson, et al.; U.S. Provisional Patent Application Ser. No. 61/058,682, filed on Mar. 26, 2009, entitled “Compositions and Methods for Diagnostics, Therapies, and other Applications,” by D. Levinson; U.S. Provisional Patent Application Ser. No. 61/163,793, filed Mar. 26, 2009, entitled “Compositions and Methods for Diagnostics, Therapies, and Other Applications,” by D. Levinson; U.S. patent application Ser. No. 12/478,756, filed Jun. 4, 2009, entitled “Compositions and Methods for Diagnostics, Therapies, and Other Applications”; International Patent Application No. PCT/US09/046333, filed Jun. 4, 2009, entitled “Compositions and Methods for Diagnostics, Therapies, and Other Applications”; U.S. Provisional Patent Application Ser. No. 61/163,710, filed Mar. 26, 2009, entitled “Systems and Methods for Creating and Using Suction Blisters or Other Pooled Regions of Fluid within the Skin”; U.S. Provisional Patent Application Ser. No. 61/156,632, filed Mar. 2, 2009, entitled “Oxygen Sensor”; U.S. Provisional Patent Application Ser. No. 61/269,436, filed Jun. 24, 2009, entitled “Devices and Techniques associated with Diagnostics, Therapies, and Other Applications, Including Skin-Associated Applications”; and U.S. Provisional Patent Application Ser. No. 61/163,791, filed on Mar. 26, 2009, entitled “Compositions and Methods for Rapid One-Step Diagnosis,” by D. Levinson; U.S. Provisional Patent Application Ser. No. 61/257,731, filed Nov. 3, 2009, entitled “Devices and Techniques Associated with Diagnostics, Therapies, and Other Applications, Including Skin-Associated Applications”; and U.S. Provisional Patent Application Ser. No. 61/294,543, filed Jan. 13, 2010, entitled “Blood Sampling Device and Method.” Also incorporated herein by reference are the following U.S. patent applications being filed on even date herewith: “Systems and Methods for Creating and Using Suction Blisters or Other Pooled Regions of Fluid within the Skin,” by Levinson, et al.; “Devices and Techniques Associated with Diagnostics, Therapies, and Other Applications, Including Skin-Associated Applications,” by Bernstein, et al.; and “Techniques and Devices Associated with Blood Sampling,” by Levinson et al.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of and “consisting essentially of” shall be closed or semi-closed transitional phrases, respectively, as set forth in the United States Patent Office Manual of Patent Examining Procedures, Section 2111.03.

What is claimed is:

1. A device able to determine localized oxygen proximate the skin when the device is applied to the skin of a subject.

2. The device of claim 1, wherein at least a portion of the device is at least partially inserted into the skin.

3. The device of claim 1, wherein the device comprises a patch.

4. The device of claim 3, wherein the patch comprises an adhesive layer.

5. The device of claim 3, wherein the patch comprises a substantially oxygen-impermeable layer.

6. The device of claim 3, wherein the patch comprises a layer comprising particles.

7. The device of claim 1, wherein the device comprises particles.

8. The device of claim 7, wherein the device consists essentially of particles.

9. The device of claim 7, wherein the device is completely insertable into the skin of the subject.

10. The device of claim 7, wherein the particles have an average diameter of less than about 1 mm.

11. The device of claim 7, wherein the particles have an average diameter of less than about 100 micrometers.

12. The device of claim 7, wherein at least some of the particles are nanoparticles.

13. The device of claim 7, wherein at least some of the particles are anisotropic.

14. The device of claim 7, wherein at least some of the particles comprise a polymer.

15. The device of claim 7, wherein at least some of the particles comprise a biodegradable polymer.

16. The device of claim 7, wherein at least some of the particles comprise a hydrogel.

17. The device of claim 7, wherein at least some of the particles comprise an semiconductive material.

18. The device of claim 7, wherein at least some of the particles are spherical.

19. The device of claim 7, wherein at least some of the particles contain at least two distinct surface regions including at least a first surface region and a second surface region.

20. The device of claim 19, wherein the first surface region comprises an oxygen-sensitive agent.

21. The device of claim 20, wherein the agent exhibits increasing aggregation with decreasing oxygen concentration.

22. The device of claim 19, wherein the first surface region comprises a protein.

23. The device of claim 22, wherein the protein is hemoglobin.

24. The device of claim 22, wherein the protein is sickle cell hemoglobin.

25. The device of claim 21, wherein the agent exhibits different degrees of polymerization when exposed to different oxygen concentrations.

26. The device of claim 21, wherein the agent exhibits at least about 10% polymerization when exposed to blood within the subject containing a concentration of oxygen less than about 90% of the saturation oxygen concentration of the blood.

27. The device of claim 1, wherein at least a portion of the device exhibits a determinable change upon a change in localized oxygen concentration.

28. The device of claim 27, wherein the determinable change is a change in color.

29. The device of claim 27, wherein the determinable change is a change in temperature.

30. The device of claim 27, wherein the determinable change is determinable by the unaided human eye.

31. The device of claim 1, wherein the device is able to determine localized oxygen in the dermis of the skin.

32. The device of claim 1, wherein the device is able to determine localized oxygen in the epidermis of the skin.

33. The device of claim 1, wherein the device is able to determine localized oxygen in interstitial fluid within the skin.

34. The device of claim 1, wherein the device is able to determine localized oxygen proximate the skin.

35. A device at least partially insertable into the skin of a subject, the device able to determine oxygen concentration proximate at least a portion of the skin of the subject.

36. A skin patch exhibiting a determinable feature responsive to oxygen when the skin patch is applied to a subject.

37. A device containing a plurality of agents that exhibit increasing aggregation with decreasing oxygen concentration.

38. An article, comprising:
a plurality of particles at least partially coated with sickle-cell hemoglobin.

39. An article, comprising:
a liquid containing a plurality of agents that are able to aggregate when the concentration of oxygen within the liquid is less than about 90% of the saturation oxygen concentration of the liquid, but are not able to substantially aggregate when the liquid is saturated with oxygen.

40. An article, comprising:
a plurality of particles coated with a polymer that exhibits at least about 10% polymerization when exposed to blood containing a concentration of oxygen less than about 90% of the saturation oxygen concentration of the blood.

41. A method, comprising:
determining blood oxygen in a subject by administering an oxygen-sensitive agent to the subject.

42. The method of claim 41, wherein the subject is suspected or at risk of having sleep apnea.

43. The method of claim 41, wherein the subject is an infant.

44. The method of claim 41, wherein the subject is suspected or at risk of having pressure ulcers or blisters.

45. The method of claim 41, wherein the subject is human.

46. The method of claim 41, wherein the subject is suspected or at risk of having bed sores.

47. The method of claim 41, wherein the oxygen-sensitive agent comprises particles.

48. The method of claim 47, wherein at least some of the particles are at least partially coated with hemoglobin.

49. The method of claim 48, wherein the hemoglobin is sickle cell hemoglobin.

50. The method of claim 41, wherein the oxygen-sensitive agent is able to determine the lowest blood oxygen concentration experienced by the subject.

51. The method of claim 41, comprising administering the oxygen-sensitive agent to the skin of the subject.

52. The method of claim 51, comprising inserting the oxygen-sensitive agent into the skin of the subject.

53. The method of claim 51, comprising inserting the oxygen-sensitive agent into the dermis of the subject.

54. The method of claim 51, comprising inserting the oxygen-sensitive agent into the epidermis of the subject.

55. The method of claim 47, comprising:
creating a suction blister in the skin of the subject; and
inserting the oxygen-sensitive agent into the suction blister.

56. The method of claim 41, wherein the oxygen-sensitive agent is applied to the skin of the subject.

57. The method of claim 56, further comprising examining the skin where the oxygen-sensitive agent was applied to determine blood oxygen in the subject.

58. The method of claim 57, further comprising identifying a color or color change in the skin where the oxygen-sensitive agent was applied to determine blood oxygen in the subject.

59. The method of claim 41, further comprising determining a change in a property of the oxygen-sensitive agent.

60. The method of claim 41, comprising determining a change in color of the oxygen-sensitive agent.

61. A method, comprising:
determining blood oxygen in a subject by applying a skin patch to the subject.

62. A method, comprising:
applying an oxygen-sensitive agent to a tissue in vitro.
